SEQUENCE LISTING (24 sequences)

SEQ ID NO: 1 - protein, 120 aa, Artificial [sequence omitted]

SEQ ID NO: 2 - protein, 5 aa, Artificial
  Synthetic Construct
DTYIH

SEQ ID NO: 3 - protein, 17 aa, Artificial
  Synthetic Construct
RIYPTNGYTRYADSVKG

SEQ ID NO: 4 - protein, 11 aa, Artificial
  Synthetic Construct
WGGDGFYAMDY

SEQ ID NO: 5 - protein, 329 aa, Artificial
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 449 aa, Artificial
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 107 aa, Artificial [sequence omitted]

SEQ ID NO: 8 - protein, 11 aa, Artificial
  Synthetic Construct
RASQDVNTAVA

SEQ ID NO: 9 - protein, 7 aa, Artificial
  Synthetic Construct
SASFLYS

SEQ ID NO: 10 - protein, 9 aa, Artificial
  Synthetic Construct
QQHYTTPPT

SEQ ID NO: 11 - protein, 107 aa, Artificial [sequence omitted]

SEQ ID NO: 12 - protein, 214 aa, Artificial
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 329 aa, Artificial
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 449 aa, Artificial
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 107 aa, Artificial
  Synthetic Construct
RTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 16 - protein, 214 aa, Artificial
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 17 - DNA, 360 bp, Artificial
  Synthetic Construct
gaggtgcagctggtggaatccggcggaggcctggtccagcctggcggatctctgcggctgtcttgcgccgcctccggcttcaacatcaaggacacctacatccactgggtccgacaggcacctggcaagggactggaatgggtggcccggatctaccccaccaacggctacaccagatacgccgactccgtgaagggccggttcaccatctccgccgacacctccaagaacaccgcctacctgcagatgaactccctgcgggccgaggacaccgccgtgtactactgctccagatggggaggcgacggcttctacgccatggactactggggccagggcaccctggtcaccgtgtctagc

SEQ ID NO: 18 - DNA, 1347 bp, Artificial
  Synthetic Construct
gaggtgcagctggtggaatccggcggaggcctggtccagcctggcggatctctgcggctgtcttgcgccgcctccggcttcaacatcaaggacacctacatccactgggtccgacaggcacctggcaagggactggaatgggtggcccggatctaccccaccaacggctacaccagatacgccgactccgtgaagggccggttcaccatctccgccgacacctccaagaacaccgcctacctgcagatgaactccctgcgggccgaggacaccgccgtgtactactgctccagatggggaggcgacggcttctacgccatggactactggggccagggcaccctggtcaccgtgtctagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 19 - DNA, 321 bp, Artificial
  Synthetic Construct
gacatccagatgacccagtccccctccagcctgtccgcctctgtgggcgacagagtgaccatcacctgtcgggcctcccaggacgtgaacaccgccgtggcctggtatcagcagaagcccggcaaggcccccaagctgctgatctactccgcctccttcctgtactccggcgtgccctcccggttctccggctccagatctggcaccgactttaccctgaccatctccagcctgcagcccgaggacttcgccacctactactgccagcagcactacaccaccccccccacctttggccagggcaccaaggtggaaatcaag

SEQ ID NO: 20 - DNA, 642 bp, Artificial
  Synthetic Construct
gacatccagatgacccagtccccctccagcctgtccgcctctgtgggcgacagagtgaccatcacctgtcgggcctcccaggacgtgaacaccgccgtggcctggtatcagcagaagcccggcaaggcccccaagctgctgatctactccgcctccttcctgtactccggcgtgccctcccggttctccggctccagatctggcaccgactttaccctgaccatctccagcctgcagcccgaggacttcgccacctactactgccagcagcactacaccaccccccccacctttggccagggcaccaaggtggaaatcaagcggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 21 - DNA, 987 bp, Artificial
  Synthetic Construct
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatgcccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 22 - DNA, 1347 bp, Artificial
  Synthetic Construct
gaggtgcagctggtggaatccggcggaggcctggtccagcctggcggatctctgcggctgtcttgcgccgcctccggcttcaacatcaaggacacctacatccactgggtccgacaggcacctggcaagggactggaatgggtggcccggatctaccccaccaacggctacaccagatacgccgactccgtgaagggccggttcaccatctccgccgacacctccaagaacaccgcctacctgcagatgaactccctgcgggccgaggacaccgccgtgtactactgctccagatggggaggcgacggcttctacgccatggactactggggccagggcaccctggtcaccgtgtctagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatgcccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 23 - DNA, 321 bp, Artificial
  Synthetic Construct
cggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtcctgcgccgactacgagaagcacaaggtgtacgcctgcgaagtgacccaccagggcctgtccagccccgtgaccaagtccttcaaccggggcgagtgc

SEQ ID NO: 24 - DNA, 642 bp, Artificial
  Synthetic Construct
gacatccagatgacccagtccccctccagcctgtccgcctctgtgggcgacagagtgaccatcacctgtcgggcctcccaggacgtgaacaccgccgtggcctggtatcagcagaagcccggcaaggcccccaagctgctgatctactccgcctccttcctgtactccggcgtgccctcccggttctccggctccagatctggcaccgactttaccctgaccatctccagcctgcagcccgaggacttcgccacctactactgccagcagcactacaccaccccccccacctttggccagggcaccaaggtggaaatcaagcggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtcctgcgccgactacgagaagcacaaggtgtacgcctgcgaagtgacccaccagggcctgtccagccccgtgaccaagtccttcaaccggggcgagtgc

(19) United States
(12) Patent Application Publication
(10) Pub. No.: US 2023/0061858 A1
(43) Pub. Date: Mar. 2, 2023

(54) TREATMENT WITH SITE SPECIFIC HER2 ANTIBODY-DRUG CONJUGATES

(71) Applicant: Pfizer Inc., New York, NY (US)

(72) Inventors: Abraham Leung, San Francisco, CA (US); Matthew Sung, Billerica, MA (US)

(21) Appl. No.: 17/786,722

(22) PCT Filed: Dec. 17, 2020
(86) PCT No.: PCT/IB2020/062123

§ 371 Date: Jun. 17, 2022

Related U.S. Application Data
(60) Provisional application No. 62/952,159, filed on Dec. 20, 2019.
(60) Provisional application No. 63/030,463, filed on May 27, 2020.

(51) Int. Cl. A61K 47/68; A61P 35/00

(52) U.S. Cl. CPC A61K 47/6855; A61K 47/6803; A61K 47/6857; A61K 47/6863; A61K 47/6889; A61P 35/00

(57) ABSTRACT

The present disclosure provides for dosing regimens for the treatment of patients with cancer, particularly a HER2-expressing cancer, with an anti-HER2 antibody-drug conjugate (ADC). The present disclosure further provides for methods for the treatment of patients with cancer in which an anti-HER2 ADC is administered. In one embodiment, the anti-HER2 ADC is T(kK183C+K290C)-vc0101 (PF-06804103), in which the antibody T(kK183C+K290C) is linked to the auristatin drug 2-methylalanyl-N-[(3R,4S,5S)-3-methoxy-1-{(2S)-2-[(1R,2R)-1-methoxy-2-methyl-3-oxo-3-{[(1S)-2 -phenyl-1-(1,3-thiazol-2-yl) ethyl]amino}propyl]pyrrolidin-1-yl}-5-methyl-1-oxoheptan-4-yl]-N-methyl-L-valinamide (also known as “0101”) via the cleavable linker maleimidocaproyl-valine-citmlline-p-aminobenzyloxycarbonyl (also known as “vc”).

### CROSS-REFERENCE TO RELATED APPLICATIONS

This application claims the benefit of U.S. Provisional Application No. 62/952,159 filed Dec. 20, 2019 and U.S. Provisional Application No. 63/030,463 filed May 27, 2020. The disclosure of each of the provisional applications is incorporated herein by reference in its entirety.

### REFERENCE TO SEQUENCE LISTING

A Sequence Listing is provided herewith as a text file, “PC72533A SEQLISTING_ST25. txt” created on Nov. 12, 2020 and having a size of 32 KB. The contents of the text file are incorporated by reference herein in their entirety.

### BACKGROUND

The present invention relates to therapeutic regimens for treatment of patients with cancer, particularly human epidermal growth factor receptor 2 (HER2)-expressing cancers. The subject therapeutic regimens involve administration of a HER2 antibody-drug conjugate (ADC) to patients in need thereof.

HER2, also known as ErbB2, p185 and CD340, is a receptor tyrosine kinase that is involved in the regulation of various cellular functions. Amplification of the gene encoding HER2 with consequent overexpression of the receptor was observed in breast and ovarian cancers and correlates with a poor prognosis (Slamon et al., 1987, Science 235(4785):177-82; Slamon et al., 1989, Science 244:707-12; Anbazhagan et al., 1991, Annals Oncology 2(1):47-53; Andrulis et al., 1998, J Clinical Oncology 16(4):1340-9). Overexpression of HER2 (frequently but not necessarily due to gene amplification) has also been observed in other tumor types including gastric, endometrial, non-small cell lung cancer, colon, pancreatic, bladder, kidney, prostate and cervical (Scholl et al., 2001, Annals Oncology 12 (Suppl. 1):S81-7; Menard et al., 2001, Ann Oncol 12(Suppl 1):515-9; Martin et al., 2014, Future Oncology 10:1469-86).

HER2-specific monoclonal antibodies have been approved for treating HER2-positive cancers, such as trastuzumab and pertuzumab. Trastuzumab (trade name Herceptin) is a humanized monoclonal antibody that binds to the extracellular domain of HER2 (Carter et al. 1992, PNAS 89:4285-9 and U.S. Pat. No. 5,821,337). Trastuzumab was approved for the treatment of patients with metastatic breast cancer whose tumors overexpress the HER2 protein. Although trastuzumab is a breakthrough in treating patients with HER2-overexpressing breast cancers that have received extensive prior anti-cancer therapy, segments of patients in this population fail to respond, respond only poorly or become resistant to trastuzumab treatment. Trastuzumab has also been approved by regulatory agencies for treating HER2-positive gastric cancer: trastuzumab. However, that approval was for combination therapy with cisplatin and a fluoropyrimidine (chemotherapy) and there was only an increase in median survival of 2 months over chemotherapy alone. Pertuzumab (also called 2C4, trade name Perjeta) is a monoclonal antibody used in combination with trastuzumab and docetaxel for the treatment of metastatic HER2-positive breast cancer. It is also used in the same combination as a neoadjuvant in early HER2-positive breast cancer

Although these HER2-targeting therapies have transformed the clinical practice for HER2-positive breast cancer and have resulted in survival benefits, not all patients respond to the therapies. Moreover, the vast majority of patients who initially respond to the treatment will eventually relapse. This is thought to be due to the high degree of intratumoral heterogeneity of HER2 expression in breast cancer and lack of efficacy of current anti-HER2 therapeutics in tumor cells expressing relatively low levels of HER2. A great deal of effort has been put into developing better anti-HER2 agents that can kill cancer cell populations expressing a broad range of HER2. Given the lack of clinical success in developing therapies to treat tumors with relatively low levels of HER2, this remains an area of high unmet medical need.

ADCs are a class of drugs that use antibodies specifically targeting tumor-associated antigens as vehicles to deliver covalently attached small-molecule toxins into cancer cells. Trastuzumab emtansine (also known as ado-trastuzumab emtansine, trastuzumab-DM1, or T-DM1; trade name Kadcyla®) is an antibody drug conjugate consisting of trastuzumab conjugated to the maytansinoid agent DM1 via the stable thioether linker MCC (4-[N-maleimidomethyl] cyclohexane-1-carboxylate) (Lewis et al., 2008, Cancer Res. 68:9280-90;Krop et al., 2010, J Clin Oncol. 28:2698-2704; U.S. Pat. No. 8,337,856). It was approved for the treatment of HER2 positive metastatic breast cancer in patients who had been previously treated with trastuzumab and a taxane drug and became trastuzumab refractory. As seen with trastuzumab, there are segments of the patients in the HER2-overexpressing breast cancer population that do not experience successful long-term therapy with trastuzumab emtansine.

Therefore, there is a significant clinical need for further HER2-directed cancer therapies for those patients with HER2-overexpressing tumors or other diseases associated with HER2 overexpression that do not respond, respond poorly, or become resistant to trastuzumab and/or trastuzumab emtansine treatment.

### SUMMARY

The present disclosure provides dosing regimens for the treatment or prophylaxis of cancer, such as a HER2-expressing cancer, with an anti-HER2 antibody-drug conjugate (ADC) comprising an anti-HER2 antibody linked to an anti-cancer drug. In some aspects of the invention, a dosage regimen comprises administering an effective amount of an anti-HER2 ADC to a patient at least twice every week, at least weekly (QW), at least every 2 weeks (Q2W), at least every 3 weeks (Q3W) or at least every 4 weeks (Q4W). In some particular aspects, the present disclosure provides a dosage regimen that comprises administering an effective amount of an anti-HER2 ADC to a patient every 3 weeks (Q3W).

The present disclosure also provides methods for the treatment or prophylaxis of cancer, such as a HER2-expressing cancer, comprising administering to a patient an effective amount of an anti-HER2 antibody-drug conjugate. In some aspects, the method comprises administering to the patient an effective amount an anti-HER2 antibody-drug conjugate at least twice every week, at least weekly (QW), at least every 2 weeks (Q2W), at least every 3 weeks (Q3W) or at least every 4 weeks (Q4W). In some particular aspects, the method comprises administering to the patient an effective amount of an anti-HER2 antibody-drug conjugate (ADC) every 3 weeks (Q3W).

The present disclosure also provides anti-HER2 ADCs for use in the treatment or prophylaxis of cancer, such as HER2-expressing cancers. The present disclosure also provides uses of an anti-HER2 ADC in the treatment or prophylaxis of cancer and/or a HER2-expressing cancer. The present disclosure also provides uses of an anti-HER2 ADC in the manufacture of a medicament for treatment or prophylaxis of cancer, such as a HER2-expressing cancer. The present disclosure also provides pharmaceutical compositions comprising an anti-HER2 ADC for use in the treatment or prophylaxis of a cancer, such as a HER2-expressing cancer.

In some aspects of the invention, administration of, or use of, a pharmaceutical composition or formulation comprising an anti-HER2 antibody-drug conjugate is contemplated.

The present disclosure also provides anti-HER2 ADCs formulated as a pharmaceutical composition. The present disclosure also provides methods of preparing and manufacturing anti-HER2 ADCs and pharmaceutical compositions comprising the same. The present disclosure also provides articles of manufacture and kits comprising the pharmaceutical compositions disclosed herein.

In some aspects of the invention, the anti-HER2 ADC is administered at a dose of about 0.10 mg/kg to about 10 mg/kg or any range of dosages between these values. In another aspect of the invention, the anti-HER2 ADC is administered at a dose of about 0.10 mg/kg to about 5 mg/kg, about 0.10 mg/kg to about 1 mg/kg, or about 0.10 mg/kg to about 0.50 mg/kg. In some aspects of the invention, the anti-HER2 ADCs is administered at a dose of at least 0.10, 0.15, 0.20, 0.25, 0.30, 0.35, 0.40, 0.45, 0.50, 0.55, 0.60, 0.65, 0.70, 0.75, 0.80, 0.95, 1.00, 1.10, 1.20, 1.30, 1.40, 1.50, 2.00, 2.50, 3.00, 3.50, 4.00, 4.50, 5.00, 5.50, 6.00 mg/kg. In some aspects of the invention, dosages of about 0.15 mg/kg, 0.50 mg/kg, 1.20 mg/kg, 2.00 mg/kg, 3.00 mg/kg, 4.00 mg/kg, 5.00 mg/kg, or 6.00 mg/kg are particularly contemplated. In a particular aspect of the invention, the anti-HER2 ADC is administered every 3 weeks (Q3W) at a dose of about 0.15 mg/kg, 0.50 mg/kg, 1.20 mg/kg, 2.00 mg/kg, 2.70 mg/kg, 3.00 mg/kg, 4.00 mg/kg, 5.00 mg/kg, or 6.00 mg/kg.

In some aspects of the invention, the anti-HER2 ADCs of the present disclosure comprise an antibody comprising three CDRs from a heavy chain variable region (VH) having the amino acid sequence shown in SEQ ID NO: 1 and three CDRs from a light chain variable region (VL) having the amino acid sequence shown in SEQ ID NO: 7. In another aspect of the invention, anti-HER2 ADCs comprise an antibody comprising a VH CDR1 having the amino acid sequence shown in SEQ ID NO: 2, VH CDR2 having the amino acid sequence shown in SEQ ID NO: 3, and VH CDR3 having the amino acid sequence shown in SEQ ID NO: 4, and/or VL CDR1 having the amino acid sequence shown in SEQ ID NO: 8, VL CDR2 having the amino acid sequence shown in SEQ ID NO: 9, and VL CDR3 having the amino acid sequence shown in SEQ ID NO: 10. In some aspects of the invention, the anti-HER2 ADCs comprise an antibody comprising a heavy chain protein having the amino acid sequence shown in SEQ ID NO: 14 and a light chain protein having the amino acid sequence shown in SEQ ID NO: 16. In a particular aspect of the invention, the anti-HER2 ADC comprises an antibody designated T(kK183C+K290C), which is described in U.S. Patent Publication No. 2017/0151341 and International Patent Application Publication WO 2017/093844, each of which is herein incorporated by reference in its entirety. In some embodiments, the anti-cancer drug of the ADC is the auristatin drug 2-methylalanyl-N-[(3R,4S,5S)-3-methoxy-1-{(2S) [(1R,2R)-1-methoxy-2-methyl-3-oxo-3-{ [(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as “0101”)) (Table 2 infra). In other embodiments, the antibody is linked to the anti-cancer drug via a linker. In a particular embodiment, the linker is the cleavable linker maleimidocaproyl-valine-citrulline-p-aminobenzyloxycarbonyl (also known as “vc”) (Table 2 infra). In a particular embodiment, the anti-HER2 ADC is T(kK183C+K290C)-vc0101 ADC (see FIG. 1).

In some aspects of the invention, the HER2-expressing cancer to be treated with the HER2 ADCs of the invention can express HER2 at a high, moderate or low level. In some embodiments, the cancer to be treated is resistant to, refractory to and/or relapsed from treatment with trastuzumab and/or trastuzumab emtansine (T-DM1) either of which alone or in combination with a taxane. Cancers to be treated include, but are not limited to, breast cancer, ovarian cancer, lung cancer, gastric cancer, esophageal cancer, colorectal cancer, urothelial cancer, pancreatic cancer, salivary gland cancer and brain cancer or metastases of the aforementioned cancers. In a more specific embodiment, the breast cancer is hormone receptor positive breast cancer, estrogen receptor and progesterone receptor negative breast cancer or triple negative breast cancer (TNBC). In another embodiment, the lung cancer is non-small cell lung cancer (NSCLC).

### BRIEF DESCRIPTION OF THE DRAWINGS

FIG. 1 provides the structure of the anti-HER2 immunoglobulin G1 ADC, T(kK183C+K290C)-vc0101, which comprises the anti-HER2 antibody T(kK183C+K290C) and 0101 payload with vc linker. Each black circle represents a linker/payload that is conjugated to the monoclonal antibody. The underlined entity is supplied by the amino acid residue on the antibody through which conjugation occurs.

FIG. 2 provides the overall clinical study design for the ADC, T(kK183C+K290C)-vc0101 (PF-06804103).

FIG. 3 provides the best percent change in tumor size in response-evaluable patients with gastric and esophageal junction cancer or breast cancer administered T(kK183C+K290C)-vc0101 (also referred to herein as “PF-06804103”). Based on RECIST criteria. Two response-evaluable patients with only non-target lesions are not included. Legend: θ=breast cancer; □=gastric and esophageal junction cancer; and for PF-06804103 Treatment Groups: A=0.15 mg/kg; B=0.5 mg/kg; C=1.2 mg/kg; D=2.0 mg/kg; E=3.0 mg/kg; F=4.0 mg/kg; and G=5.0 mg/kg.

FIGS. 4A and 4B provide PK profile for the ADC T(kK183C+K290C)-vc0101 (PF-06804103) (FIG. 4A) and the Unconjugated payload (0101) (FIG. 4B) during Cycle 1. Legend for PF-06804103 Treatment Groups: A=0.15 mg/kg; B=0.5 mg/kg; C=1.2 mg/kg; D=2.0 mg/kg; E=3.0 mg/kg; F=4.0 mg/kg; and G=5.0 mg/kg.

### DETAILED DESCRIPTION

The present disclosure provides dosing regimens for the treatment or prophylaxis of cancer and/or a HER2-expressing cancer with an anti-HER2 ADC or a pharmaceutical composition comprising the same. In some aspects of the invention, a dosage regimen comprises administering an effective amount of an anti-HER2 ADC to a patient at least twice every week, at least weekly (QW), at least every 2 weeks (Q2W), at least every 3 weeks (Q3W) or at least every 4 weeks (Q4W). In some particular aspects of the invention, a dosage regimen may comprise administering an effective amount of an anti-HER2 ADC to a patient every 3 weeks (Q3W). In some particular aspects of the invention, the efficacy of the dosage regimen may be determined by measuring the decrease in tumor size as compared to the tumor size in the patient prior to the initial administration of the anti-HER2 ADC. For example, the tumor may decrease in size by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100%, or up to a point at which the tumor is no longer detectable. The present disclosure also provides methods for the treatment or prophylaxis of cancer and/or a HER2-expressing cancer comprising administering an anti-HER2 ADC or pharmaceutical composition comprising the same to a patient. The present disclosure further provides methods for the treatment or prophylaxis of cancer and/or a HER2-expressing cancer in which an anti-HER2 ADC or pharmaceutical composition comprising the same is intravenously administered to a patient every 3 weeks (Q3W).

The present disclosure also provides anti-HER2 ADCs and pharmaceutical compositions comprising the same for use in the treatment or prophylaxis of cancer and/or a HER2-expressing cancer. The present disclosure further provides anti-HER2 ADCs or pharmaceutical compositions comprising the same for use in the treatment or prophylaxis of cancer and/or a HER2-expressing cancer in which the anti-HER2 ADC or pharmaceutical composition comprising the same is intravenously administered to a patient every 3 weeks (Q3W).

The present disclosure also provides uses of an anti-HER2 ADC or pharmaceutical composition comprising the same for use in the dosing regimen, treatment, or prophylaxis of cancer and/or a HER2-expressing cancer. The present disclosure further provides uses of an anti-HER2 ADC or pharmaceutical composition comprising the same for treatment or prophylaxis of cancer and/or a HER2-expressing cancer in which an anti-HER2 ADC or pharmaceutical composition comprising the same is intravenously administered to a patient every 3 weeks (Q3W).

The present disclosure also provides uses of an anti-HER2 ADC in the manufacture of a medicament for treatment or prophylaxis of a cancer and/or a HER2-expressing cancer. The present disclosure also provides pharmaceutical compositions comprising an anti-HER2 ADC for use in the treatment or prophylaxis of cancer and/or a HER2-expressing cancer.

The present disclosure also provides anti-HER2 ADCs and pharmaceutical compositions comprising the same for use in the treatment or prophylaxis of a condition associated with HER2 expression in a patient. The conditions associated with HER2 expression include, but are not limited to, abnormal HER2 expression, altered or aberrant HER2 expression, HER2 overexpression, and a proliferative disorder (e.g., cancer).

The present disclosure also provides methods for the treatment or prophylaxis of a condition associated with HER2 expression in a patient comprising administering an anti-HER2 ADC or pharmaceutical composition comprising the same to the patient.

The present disclosure also provides uses of an anti-HER2 ADC or pharmaceutical composition comprising the same for treatment or prophylaxis of a condition associated with HER2 expression in a patient.

The present disclosure also provides uses of an anti-HER2 ADC in the manufacture of a medicament for treatment or prophylaxis of a condition associated with HER2 expression in a patient.

The present invention also provides pharmaceutical compositions for use in the treatment or prophylaxis of a condition associated with HER2 expression in a patient.

The present disclosure also provides anti-HER2 ADCs and pharmaceutical compositions comprising the same for use in inhibiting growth or progression of a HER2-expressing tumor in a patient.

The present disclosure also provides methods for inhibiting growth or progression of a HER2-expressing tumor in a patient comprising administering an anti-HER2 ADC or pharmaceutical composition comprising the same to the patient.

The present disclosure also provides uses of an anti-HER2 ADC or pharmaceutical composition comprising the same for inhibiting growth or progression of a HER2-expressing tumor in a patient.

The present disclosure also provides uses of an anti-HER2 ADC in the manufacture of a medicament for inhibiting growth or progression of a HER2-expressing tumor.

The present disclosure also provides pharmaceutical compositions comprising an anti-HER2 ADC for use in inhibiting growth or progression of an HER2-expressing tumor.

The present disclosure also provides anti-HER2 ADCs and pharmaceutical compositions comprising the same for use in inhibiting metastasis of HER2-expressing cancer cells in a patient.

The present disclosure also provides methods for inhibiting metastasis of HER2-expressing cancer cells in a patient comprising administering an anti-HER2 ADC or pharmaceutical composition comprising the same to the patient.

The present disclosure also provides uses of an anti-HER2 ADC or pharmaceutical composition comprising the same for inhibiting metastasis of HER2-expressing cancer cells in a patient.

The present disclosure also provides uses of an anti-HER2 ADC in the manufacture of a medicament for inhibiting metastasis of HER2-expressing cancer cells.

The present disclosure also provides pharmaceutical compositions comprising an anti-HER2 ADC for use in inhibiting metastasis of HER2-expressing cancer cells.

The present disclosure also provides anti-HER2 ADCs and pharmaceutical compositions comprising the same for use in inducing regression of a HER2-expressing tumor in a patient.

The present disclosure also provides methods for inducing regression of a HER2-expressing tumor in a patient comprising administering an anti-HER2 ADC or pharmaceutical composition comprising the same to the patient.

The present disclosure also provides uses of an anti-HER2 ADC or pharmaceutical composition comprising the same for inducing regression of a HER2-expressing tumor in a patient.

The present disclosure also provides uses of an anti-HER2 ADC in the manufacture of a medicament for inducing regression of a HER2-expressing tumor.

The present disclosure also provides pharmaceutical compositions comprising an anti-HER2 ADC for use in inducing regression of a HER2-expressing tumor.

The present disclosure also provides anti-HER2 ADCs formulated as a pharmaceutical composition. The present disclosure also provides methods of preparing and manufacturing anti-HER2 ADCs and pharmaceutical compositions comprising the same. The present disclosure also provides articles of manufacture and kits comprising the pharmaceutical compositions disclosed herein.

### GENERAL TECHNIQUES

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Gellis, ed., 1998) Academic Press; Animal Cell Culture (R I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); lmmunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995).

As used herein, the terms “antibody-drug conjugate” or “ADC” refers to a molecule composed of an antibody linked to an anti-cancer drug. The antibody specifically binds to a certain tumor antigen, such as HER2. The antibodies used in an ADC may be full-length antibodies, antigen-binding fragments of a full-length antibody, or antibody derivatives. Typically, the anti-cancer drug is conjugated to the antibody via a linker. Thus, in one embodiment, the ADC provided by the present disclosure comprises an antibody, or antigen-binding fragment thereof, that binds to HER2, and a linker-drug moiety.

As used herein, the term “HER2” refers to a transmembrane tyrosine kinase receptor that belongs to the EGFR family. The wild type human HER2 protein is described, for example, in Semba et al., 1985, PNAS 82:6497-6501 and Yamamoto et al., 1986, Nature 319:230-4 and Genbank Accession Number X03363. The term “HER2” includes variants, isoforms, homologs, orthologs and paralogs. In some aspects of the invention, antibodies and antibody-drug conjugates cross-react with HER2 from species other than human, such as HER2 of mouse, rat, or primate, as well as different forms of HER2 (e.g., glycosylated HER2). In other aspects, the antibodies and antibody-drug conjugates may be completely specific for human HER2 and may not exhibit species or other types of cross-reactivity. As used herein the term HER2 refers to naturally occurring human HER2 unless contextually dictated otherwise. Therefore, a “HER2 antibody”, “anti-HER2 antibody”, or other similar designation, means an antibody that associates, binds, or reacts with the HER2 type ligand or isoform, or fragment or derivative thereof. Further, a “HER2 antibody-drug conjugate”, “anti-HER2 antibody-drug conjugate” refers to an antibody-drug conjugate or ADC (as defined herein) that comprises an anti-HER2 antibody as defined herein.

In some embodiments, the antibody used in the present invention specifically binds to HER2. In a specific embodiment, the HER2 antibody binds to the same epitope on HER2 as trastuzumab. In a more specific embodiment, the HER2 antibody has the same variable region CDRs as trastuzumab. In yet a more specific embodiment, the HER2 antibody has the same variable regions (i.e., V_{H} and V_{L}) as trastuzumab.

As used herein, the term “linker” refers to a chemical moiety that joins the antibody to the drug payload. Attachment of a linker to an antibody can be accomplished in a variety of ways, such as through surface lysines, reductive-coupling to oxidized carbohydrates, cysteine residues liberated by reducing interchain disulfide linkages, reactive cysteine residues engineered at specific sites, and acyl donor glutamine-containing tag or an endogenous glutamine made reactive by polypeptide engineering in the presence of transglutaminase and an amine. The present invention uses site specific methods to link the antibody to the drug payload. In one embodiment, conjugation occurs through cysteine residues that have been engineered into the antibody constant region. In another embodiment, conjugation occurs through acyl donor glutamine residues that have either been a) added to the antibody constant region via a peptide tag, b) engineered into the antibody constant region or c) made accessible/reactive by engineering surrounding residues. Linkers can be cleavable (i.e., susceptible to cleavage under intracellular conditions) or non-cleavable. In some embodiments, the linker is a cleavable linker. In some particular embodiments, the linker of the HER2 ADC is maleimidocaproyl-valine-citrulline-p-aminobenzyloxycarbonyl (hereinafter “vc”).

As used herein, the terms “anti-cancer drug,” “drug”, “payload” and “drug payload,” which are used interchangeably, refer to a therapeutic agent useful in treating cancer, such as cytotoxic agents, chemotherapeutic agents, cytostatic agents, and immunomodulatory agents. In some embodiments, the drug is preferably membrane permeable. In some embodiments, therapeutic agents have a cytotoxic effect on tumors including the depletion, elimination and/or the killing of tumor cells. In a specific embodiment, the drug is an anti-mitotic agent. In a more specific embodiment, the drug is an auristatin. Examples of anti-cancer drugs in the ADC include 2-methylalanyl-N-[(3R,4 S,5 S)-3-methoxy-1-{(2 S)-2 -[(1R,2R)-1 -methoxy-2-methyl-3-oxo-3-{[(1S)-2-phenyl-1-(1,3-thiazol -2-yl)ethyl]amino}propyl]pyrrolidin-1-yl}-5-methyl-1-oxoheptan-4-yl]-N-methyl-L-valinamide (also known as 0101), 2-methylalanyl-N-[(3R,4S,5S)-1-{(2S)-2-[(1R,2R)-3-{[(1S)-1-carboxy-2-phenylethyl]amino}-1-methoxy -2-methyl-3-oxopropyl]pyrrolidin-1-yl}-3-methoxy-5-methyl-1-oxoheptan -4-yl]-N-methyl-L-valinamide (also known as 8261), 2-methyl-L-prolyl-N-[(3R,4 S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl -1-oxoheptan-4-yl]-N-methyl-L-valinamide, trifluoroacetic acid salt (also known as 6121), 2-methylalanyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl-5-methyl-1-oxoheptan-4-yl]-N-methyl-L-valinamide (also known as 8254), 2-methylalanyl-N-[3R,4S,5S)-1-{(2S)-2-[(1R,2R)-3-{[(1S,2R)-1-hydroxy-1- phenylpropan-2-yl]amino}-1-methoxy-2-methyl-3-oxopropyl]pyrrolidin-1-yl}-3- methoxy-5-methyl-1-oxoheptan-4-yl]-N-methyl-L-valinamide (also known as 6780), 2-methyl-L-prolyl-N-[(3R,4S,5 S)-1-{(2S)-2-[(1R,2R)-3-{[(1 S)-1-carboxy-2-phenylethyl]amino}-1-methoxy -2-methyl-3-oxopropyl]pyrrolidin-1-yl}-3-methoxy-5-methyl-1-oxoheptan-4-yl]-N-methyl-L-valinamide, trifluoroacetic acid salt (also known as 0131), N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as MMAD), N-methyl-L-valyl-N-[(3R,4S,5S)-1-{(2S)-2[(1R,2R)-3[(1S,2R)- 1-hydroxy-1-phenylpropan-2-yl]amino}1-methoxy-2-methyl-3-oxopropyl]pyrrolidin-1-yl}-3-methoxy-5-methyl-1-oxoheptan-4-yl]-N-methyl-L-valinamide (also known as MMAE), and N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide (also known as MMAF). In a yet more specific embodiment, the drug is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as 0101).

As used herein, the term “linker-drug moiety” refers to the molecule resulting from a drug linked or conjugated to a linker.

As used herein, the terms “binding affinity” or “K_{D}” refers to the equilibrium dissociation constant of a particular antigen-antibody interaction. The K_{D} is the ratio of the rate of dissociation, also called the “off-rate” or “k_{d}”, to the rate of association, or “on-rate” or “kₐ”. Thus, KD equals k_{d}/kₐ and is expressed as a molar concentration (M). It follows that the smaller the K_{D}, the stronger the binding affinity. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance, typically using a biosensor system such as a BIACORE® system.

An “antibody” or “Ab” is an immunoglobulin molecule capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” encompasses any type of antibody, including but not limited to monoclonal antibodies, polyclonal antibodies, antigen-binding fragments (or portion), such as Fab, Fab′, F(ab′3 )₂, Fd, Fv, Fc, etc., of intact antibodies that retain the ability to specifically bind to a given antigen (e.g. HER2), an isolated complementarity determining region (CDR), bispecific antibodies, heteroconjugate antibodies, mutants thereof, fusion proteins having an antibody, or antigen-binding fragment thereof, (e.g., a domain antibody), single chain (ScFv) and single domain antibodies (e.g., shark and camelid antibodies), maxibodies, minibodies, intrabodies, diabodies, triabodies, tetrabodies, v-NAR and bis-scFv (see, e.g., Holliger and Hudson, 2005, Nature Biotechnology 23(9): 1126-1136), humanized antibodies, chimeric antibodies and any other modified configuration of the immunoglobulin molecule that includes an antigen recognition site of the required specificity, including glycosylation variants of antibodies, amino acid sequence variants of antibodies, and covalently modified antibodies. The antibodies may be of murine, rat, human, or any other origin (including chimeric or humanized antibodies). In some aspects of the invention, the antibody, or antigen-binding fragment thereof, of the disclosed anti-HER2 antibody-drug conjugates is a chimeric, humanized, or a recombinant human antibody, or HER2-binding fragment thereof.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Lazikani et al., J. Molec. Biol. 273:927-948 (1997)). As used herein, a CDR may refer to CDRs defined by either approach or by a combination of both approaches.

A CDR of a variable domain are comprised of amino acid residues within the variable region that are identified in accordance with the definitions of Kabat, Chothia, the accumulation of both Kabat and Chothia, VBASE2, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, (1989). The CDR positions may also be derived from an analysis of the VBASE2 database. (See, e.g. Retter et al., Nucleic Acids Res. 33(Database Issue): D671-D674, 2005).

Other approaches to CDR identification include the “AbM definition,” which is a compromise between Kabat and Chothia and is derived using Oxford Molecular's AbM antibody modeling software (now ACCELRYS®), or the “contact definition” of CDRs based on observed antigen contacts, set forth in MacCallum et al., J. Mol. Biol., 262:732-745, (1996). In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding As used herein, a CDR may refer to CDRs defined by any approach known in the art, including combinations of approaches. The methods used herein may utilize CDRs defined according to any of these approaches. For anti-HER2 antibody-drug conjugates described herein, CDRs may be defined in accordance with any of Kabat, Chothia, extended, VBASE2, AbM, contact, and/or conformational definitions.

Antibodies, antibody domains, and antigen-binding fragments thereof may be described as “polypeptides”, “oligopeptides”, “peptides” and “proteins”, i e , chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains. Amino acids may be referred to herein by either their commonly known three letter symbols or by the one-letter symbols recommended by the IUPAC-IUB Commission on Biochemical Nomenclature.

As used herein, “humanized antibody” or “CDR grafted antibody” refers to forms of non-human (e.g. murine) antibodies that are chimeric immunoglobulins, immunoglobulin chains, or fragments thereof (such as Fv, Fab, Fab′, F(ab′)₂ or other antigen binding subsequences of antibodies) that contain minimal sequences derived from a non-human immunoglobulin. Preferably, humanized antibodies are human immunoglobulins (recipient antibody) in which residues from one or more complementarity determining regions (CDRs) of the recipient are replaced by residues from one or more CDRs of a non-human species (donor antibody) such as mouse, rat, or rabbit having the desired specificity, affinity, and capacity.

As used herein, the term “dosing regimen” refers to the total course of treatment administered to a patient, e.g., treatment with an anti-HER2 ADC.

As used herein, “dose limiting toxicity” (DLT) refers to the dosage of the anti-HER2 antibody-drug conjugate that is contraindicative of a further increase in dosage. DLT is graded according to NCI Common Terminology Criteria (v 4.03) during the first cycle of treatment which is not clearly and incontrovertibly due to underlying disease/progression or extraneous cause. Hematologic: grade 4 neutropenia for >7 days; febrile neutropenia; grade ≥3 neutropenia with infection; thrombocytopenia with clinically significant bleeding; or grade 4 thrombocytopenia. Non-hematologic: grade ≥3 toxicities, that are considered clinically significant, excluding nausea, vomiting or diarrhea or electrolyte abnormality lasting <72 hours, that does not resolve spontaneously or does not respond to conventional medical interventions or other supportive care; or delay by more than 2 weeks in receiving the next scheduled cycle due to persisting toxicities.

As used herein “maximum tolerated dose” (MTD) refers to the highest dosage of the anti-HER2 antibody-drug conjugate that does not cause unacceptable side effects or intolerable toxicities. MTD is estimated using the mTPI based on observed DLT rate, with a target DLT rate of 27.5% and equivalence interval of 22.5-32.5%. At least 9 patients will be accumulated at a dose that is predicted to be the MTD.

In the dosing regimen or method provided by the present disclosure, the anti-HER2 ADC may be administered as an initial treatment of a condition, or for treatment of conditions that are unresponsive to conventional therapies. The term “conventional therapies” refer to treatments that are widely accepted and used by healthcare professionals. Examples of conventional therapy for cancer include chemotherapy, radiation therapy, and surgery. In addition, the HER2 ADC may be used in combination with other therapies (e.g., surgical excision, radiation, additional anti-cancer drugs, etc.) to thereby elicit additive or potentiated therapeutic effects and/or reduce toxicity of some anti-cancer agents. The HER2 ADCs used in the regimens or methods provided by the present disclosure may be co-formulated with additional agents for co-administration, or formulated separately with additional agents for separate administration in any order.

As used herein, the phrases “effective amount” or “effective dosage” are used interchangeably and refer to an amount of a drug (e.g., anti-HER2 ADC), compound, or pharmaceutical composition necessary to achieve one or more beneficial or desired prophylactic or therapeutic results. For prophylactic use, beneficial or desired results include eliminating or reducing the risk of developing a disease (e.g., cancer and/or HER2-expressing cancer), delaying the onset of the disease, or preventing the progression of the disease. For therapeutic use, beneficial or desired results include eliminating, reducing the incidence of, or ameliorating one or more symptoms of, these diseases or conditions. Determination of an effective amount or dosage may include observing or measuring changes in: biochemical or histological markers; behavioral symptoms of the disease; complications of the disease; and intermediate pathological phenotypes presenting during development of the disease. Determination of an effective amount or dosage may also include observing or measuring a decrease in the dose of another drug/medication required to treat the disease; or an increase in the efficacy of another drug/medication. In particular aspects of the invention, the efficacy of treatment may be determined by measuring the decrease in tumor size as compared to the tumor size in the patient prior to the initial administration of the anti-HER2 ADC using methods known in the art (e.g., Response Evaluation Criteria In Solid Tumors (RECIST)). For example, the tumor may decrease in size by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or up to 100% or up to a point at which the tumor is no longer detectable.In one aspect, the disclosure provides a method for treating a condition associated with HER2 expression in a patient. The disclosure also provides an ADC, or a pharmaceutical composition, as described herein, for use in a method for treating a condition associated with HER2 expression in a patient. The disclosure further provides the use of an ADC, or a pharmaceutical composition, as described herein, in the manufacture of a medicament for treating a condition associated with HER2 expression in a patient.

In some aspects of the disclosure, the method of treating a condition associated with HER2 expression in a patient includes administering to the patient in need thereof an effective amount of a composition (e.g., pharmaceutical composition) comprising a HER2 ADC as described herein. The conditions associated with HER2 expression include, but are not limited to, abnormal HER2 expression, altered or aberrant HER2 expression, HER2 overexpression, and a proliferative disorder (e.g., cancer).

In some aspects of the invention, the HER2-expressing cancer to be treated with the site specific HER2 ADCs of the invention can express HER2 at a high, moderate or low level. In some embodiments, the cancer to be treated is resistant to, refractory to and/or relapsed from treatment with trastuzumab and/or trastuzumab emtansine (T-DM1) either of which alone or in combination with a taxane. Cancers to be treated include, but are not limited to, breast cancer, ovarian cancer, lung cancer, gastric cancer, esophageal cancer, colorectal cancer, urothelial cancer, pancreatic cancer, salivary gland cancer and brain cancer or metastases of the aforementioned cancers. In a more specific embodiment, the breast cancer is hormone receptor positive breast cancer, estrogen receptor and progesterone receptor negative breast cancer or triple negative breast cancer (TNBC). In another embodiment, the lung cancer is non-small cell lung cancer (NSCLC).

In some aspects, the present disclosure provides for a method of inhibiting tumor growth or progression in a patient who has a HER2 expressing tumor, including administering to the patient in need thereof an effective amount of a composition having the HER2 ADCs as described herein. In other aspects of the invention, provided is a method of inhibiting metastasis of HER2 expressing cancer cells in a patient, including administering to the patient in need thereof an effective amount of a composition having the HER2 ADCs as described herein. In other aspects of the invention, provided is a method of inducing regression of a HER2 expressing tumor regression in a patient, including administering to the patient in need thereof an effective amount of a composition having the HER2 ADCs as described herein. In other aspects, the disclosure provides a HER2 ADC, or a pharmaceutical composition, as described herein, for use in a method as described above. In other aspects, the disclosure provides the use of a HER2 ADC, or a pharmaceutical composition, as described herein, in the manufacture of a medicament for use in the methods described above. The HER2 ADC may be administered according to a dosing regimen described herein.

As used herein, the terms “individual”, “subject”, and “patient” are used interchangeably and refer to a mammal, including, but not limited to, humans, non-human primates, horses, dogs, cats, mice, and rats. In a preferred aspect of the invention, the mammal is a human.

As used herein, the terms “pharmaceutically acceptable carrier” and “pharmaceutical acceptable excipient” are used interchangeably and refer to any material which, when combined with an active ingredient, allows the ingredient to retain biological activity and is non-reactive with the patient's immune system. Examples include standard pharmaceutical carriers such as a phosphate buffered saline solution, water, emulsions such as oil/water emulsion, and various types of wetting agents. Compositions comprising such carriers are formulated by well-known conventional methods (see, for example, Remington's Pharmaceutical Sciences, 18^{th} edition, A. Gennaro, ed., Mack Publishing Co., Easton, PA, 1990; and Remington, The Science and Practice of Pharmacy, 20th Ed., Mack Publishing, 2000).

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of and/or ” consisting essentially of are also provided.

Additional scientific and technical terms used in connection with the present invention, unless indicated otherwise herein, shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics, and protein and nucleic acid chemistry and hybridization described herein are those well-known and commonly used in the art.

### DOSING REGIMENS AND METHODS OF TREATMENT

The present disclosure provides for dosing levels, dosing regimens, and methods for the treatment of patients with cancer and/or an HER2-expressing cancer with an anti-HER2 antibody-drug conjugate (ADC). The present disclosure further provides for dosing levels, dosing regimens, and methods for the treatment of patients with cancer and/or a HER2-expressing cancer in which an anti-HER2 ADC is administered to a patient intravenously, subcutaneously, intramuscularly, by bolus injection, intracerebrally or by sustained release. The present disclosure further provides for dosing levels, dosing regimens and methods for the treatment of patients with cancer and/or a HER2-expressing cancer in which an anti-HER2 ADC administered to a patient at least twice every week, at least weekly (QW), at least every 2 weeks (Q2W), at least every 3 weeks (Q3W) or at least every 4 weeks (Q4W). The present disclosure further provides for dosing levels, dosing regimens and methods for the treatment of patients with cancer and/or a HER2-expressing cancer in which an anti-HER2 ADC is administered to a patient intravenously every 3 weeks (Q3W). The danti-HER2 ADCs may be administered as an initial treatment, or for treatment of cancers that are unresponsive to conventional therapies.

In some aspects of the invention, the anti-HER2 ADC is administered or is administered at a dose of about 0.10 mg/kg to about 10 mg/kg or any range of dosages between these values. In another aspect of the invention, the anti-HER2 ADC is administered or is administrable at a dose of about 0.10 mg/kg to about 5 mg/kg, about 0.10 mg/kg to about 1 mg/kg, or about 0.10 mg/kg to about 0.50 mg/kg. In some aspects of the invention, the anti-HER2 ADCs is administered or is administrable at a dose of at least 0.10, 0.15, 0.20, 0.25, 0.30, 0.35, 0.40, 0.45, 0.50, 0.55, 0.60, 0.65, 0.70, 0.75, 0.80, 0.95, 1.00, 1.10, 1.20, 1.30, 1.40, 1.50, 2.00, 2.50, 2.70, 3.00, 3.50, 4.00, 4.50, 5.00, 5.50, 6.00 mg/kg. In some aspects of the invention, dosages of about 0.15 mg/kg, 0.50 mg/kg, 1.20 mg/kg, 2.00 mg/kg, 2.70 mg/kg, 3.00 mg/kg, 4.00 mg/kg, 5.00 mg/kg, or 6.00 mg/kg are particularly contemplated. In a particular aspect of the inventionthe anti-HER2 ADC is administered or is administrable every 3 weeks (Q3W) at a dose of about 0.15 mg/kg, 0.50 mg/kg, 1.20 mg/kg, 2.00 mg/kg, 2.70 mg/kg, 3.00 mg/kg, 4.00 mg/kg, 5.00 mg/kg, or 6.00 mg/kg.

The present disclosure further provides for dosing levels, dosing regimens and methods for the treatment of patients with cancer and/or a HER2-expressing cancer in which the treatment results in a decrease in a tumor size of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% as compared to the tumor size in the patient prior to initial administration of the anti-HER2 ADC. A decrease in tumor size may be measured or determined by any method used and accepted in the art (e.g., RECIST v.1.1).

### Anti-HER2 Antibody-Drug Conjugates (ADCs)

The invention can be practiced using, for example, an anti-HER2 ADC comprising an antibody that specifically binds to human HER2. In some aspects, the antibody comprises three CDRs (i.e., CDR1, CDR2, and CDR3) from a heavy chain protein having the amino acid sequence shown in SEQ ID NO: 14 and three CDRs (i.e., CDR1, CDR2, and CDR3) from a light chain protein having the amino acid sequence shown in SEQ ID NO: 16. In another aspect, the antibody comprises a VH CDR1 having the amino acid sequence shown in SEQ ID NO: 2, VH CDR2 having the amino acid sequence shown in SEQ ID NO: 3, and VH CDR3 having the amino acid sequence shown in SEQ ID NO: 4, and/or VL CDR1 having the amino acid sequence shown in SEQ ID NO: 8, VL CDR2 having the amino acid sequence shown in SEQ ID NO: 9, and VL CDR3 having the amino acid sequence shown in SEQ ID NO: 10.

Table 1 provides the amino acid (protein) sequences and associated nucleic acid (DNA) sequences of certain humanized HER2 antibodies that may be used in constructing the site-specific ADCs for use in the dosing regimens or methods provided by the present disclosure. The CDRs shown are defined by Kabat numbering scheme.

The antibody heavy chains and light chains shown in Table 1 have the trastuzumab heavy chain variable region (VH) and light chain variable region (VL). The heavy chain constant region and light chain constant region shown in Table 1 are derivatized from trastuzumab and contain on or more modifications (relative to the respective sequences of trastuzumab) to allow for site specific conjugation when making the ADCs used in the invention. Modifications to the amino acid sequences in the antibody constant region to allow for site specific conjugation are underlined and bolded. The nomenclature for the antibodies derivatized from trastuzumab is T (for trastuzumab) and then in parenthesis the position of the amino acid of modification flanked by the single letter amino acid code for the wild type residue and the single letter amino acid code for the residue that is now in that position in the derivatized antibody. An exception to this nomenclature is “kK183C” which denotes that position 183 on the light (kappa) chain has been modified from a lysine to a cysteine. The positions of the amino acids of modifications, such as “K290C” and “kK183C,” are numbered according to the numbering of EU index of Kabat.

TABLE 1
Sequences of Humanized HER2 Antibodies

| SEQ ID | | |
|---|---|---|
| NO. | Description | Sequence |
| 1 | Trastuzumab | EVQLVESGGGLVQPGGSLRLSCAASGFNIKDTYIHWVRQAPG |
| | VH protein | KGLEWVARIYPTNGYTRYADSVKGRFTISADTSKNTAYLQM |
| | | NSLRAEDTAVYYCSRWGGDGFYAMDYWGQGTLVTVSS |
| 2 | VH CDR1 | DTYIH |
| | protein | |
| 3 | VH CDR2 | RIYPTNGYTRYADSVKG |
| | protein | |
| 4 | VH CDR3 | WGGDGFYAMDY |
| | protein | |
| 5 | Trastuzumab | ASTKGPSVFPLAPSSKSTSGGTAALGCLVKDYFPEPVTVSWN |
| | heavy chain | SGALTSGVHTFPAVLQSSGLYSLSSVVTVPSSSLGTQTYICNV |
| | constant | NHKPSNTKVDKKVEPKSCDKTHTCPPCPAPELLGGPSVFLFPP |
| | region | KPKDTLMISRTPEVTCVVVDVSHEDPEVKFNWYVDGVEVHN |
| | protein | AKTKPREEQYNSTYRVVSVLTVLHQDWLNGKEYKCKVSNK |
| | | ALPAPIEKTISKAKGQPREPQVYTLPPSREEMTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPG |
| 6 | Trastuzumab | EVQLVESGGGLVQPGGSLRLSCAASGFNIKDTYIHWVRQAPG |
| | heavy chain | KGLEWVARIYPTNGYTRYADSVKGRFTISADTSKNTAYLQM |
| | protein | NSLRAEDTAVYYCSRWGGDGFYAMDYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPREPQVYTLPPSREEMTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPG |
| 7 | Trastuzumab | DIQMTQSPSSLSASVGDRVTITCRASQDVNTAVAWYQQKPG |
| | VL protein | KAPKLLIYSASFLYSGVPSRFSGSRSGTDFTLTISSLQPEDFAT |
| | | YYCQQHYTTPPTFGQGTKVEIK |
| 8 | VL CDR1 | RASQDVNTAVA |
| | protein | |
| 9 | VL CRD2 | SASFLYS |
| | protein | |
| 10 | VL CDR3 | QQHYTTPPT |
| | protein | |
| 11 | Trastuzumab | RTVAAPSVFIFPPSDEQLKSGTASVVCLLNNFYPREAKVQWK |
| | light chain | VDNALQSGNSQESVTEQDSKDSTYSLSSTLTLSKADYEKHKV |
| | constant | YACEVTHQGLSSPVTKSFNRGEC |
| | region protein | |
| 12 | Trastuzumab | DIQMTQSPSSLSASVGDRVTITCRASQDVNTAVAWYQQKPG |
| | light chain | KAPKLLIYSASFLYSGVPSRFSGSRSGTDFTLTISSLQPEDFAT |
| | protein | YYCQQHYTTPPTFGQGTKVEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 13 | T(K290C) | ASTKGPSVFPLAPSSKSTSGGTAALGCLVKDYFPEPVTVSWN |
| | heavy chain | SGALTSGVHTFPAVLQSSGLYSLSSVVTVPSSSLGTQTYICNV |
| | constant | NHKPSNTKVDKKVEPKSCDKTHTCPPCPAPELLGGPSVFLFPP |
| | region | KPKDTLMISRTPEVTCVVVDVSHEDPEVKFNWYVDGVEVHN |
| | protein | AKTCPREEQYNSTYRVVSVLTVLHQDWLNGKEYKCKVSNK |
| | | ALPAPIEKTISKAKGQPREPQVYTLPPSREEMTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPG |
| 14 | T(K290C) | EVQLVESGGGLVQPGGSLRLSCAASGFNIKDTYIHWVRQAPG |
| | heavy chain | KGLEWVARIYPTNGYTRYADSVKGRFTISADTSKNTAYLQM |
| | protein | NSLRAEDTAVYYCSRWGGDGFYAMDYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | CPREEQYNSTYRVVSVLTVLHQDWLNGKEYKCKVSNKALP |
| | | APIEKTISKAKGQPREPQVYTLPPSREEMTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPG |
| 15 | T(kK183C) | RTVAAPSVFIFPPSDEQLKSGTASVVCLLNNFYPREAKVQWK |
| | light chain | VDNALQSGNSQESVTEQDSKDSTYSLSSTLTLSCADYEKHKV |
| | constant | YACEVTHQGLSSPVTKSFNRGEC |
| | region | |
| | protein | |
| 16 | T(kK183C) | DIQMTQSPSSLSASVGDRVTITCRASQDVNTAVAWYQQKPG |
| | light chain | KAPKLLIYSASFLYSGVPSRFSGSRSGTDFTLTISSLQPEDFAT |
| | protein | YYCQQHYTTPPTFGQGTKVEIKRTVAAPSVFIFPPSDEQLKSG |
| | | TASVVCLLNNFYPREAKVQWKVDNALQSGNSQESVTEQDSK |
| | | DSTYSLSSTLTLSCADYEKHKVYACEVTHQGLSSPVTKSFNR |
| | | GEC |
| 17 | Trastuzumab | GAGGTGCAGCTGGTGGAATCCGGCGGAGGCCTGGTCCAGC |
| | VHDNA | CTGGCGGATCTCTGCGGCTGTCTTGCGCCGCCTCCGGCTTC |
| | | AACATCAAGGACACCTACATCCACTGGGTCCGACAGGCAC |
| | | CTGGCAAGGGACTGGAATGGGTGGCCCGGATCTACCCCAC |
| | | CAACGGCTACACCAGATACGCCGACTCCGTGAAGGGCCGG |
| | | TTCACCATCTCCGCCGACACCTCCAAGAACACCGCCTACCT |
| | | GCAGATGAACTCCCTGCGGGCCGAGGACACCGCCGTGTAC |
| | | TACTGCTCCAGATGGGGAGGCGACGGCTTCTACGCCATGG |
| | | ACTACTGGGGCCAGGGCACCCTGGTCACCGTGTCTAGC |
| 18 | Trastuzumab | GAGGTGCAGCTGGTGGAATCCGGCGGAGGCCTGGTCCAGC |
| | heavy chain | CTGGCGGATCTCTGCGGCTGTCTTGCGCCGCCTCCGGCTTC |
| | DNA | AACATCAAGGACACCTACATCCACTGGGTCCGACAGGCAC |
| | | CTGGCAAGGGACTGGAATGGGTGGCCCGGATCTACCCCAC |
| | | CAACGGCTACACCAGATACGCCGACTCCGTGAAGGGCCGG |
| | | TTCACCATCTCCGCCGACACCTCCAAGAACACCGCCTACCT |
| | | GCAGATGAACTCCCTGCGGGCCGAGGACACCGCCGTGTAC |
| | | TACTGCTCCAGATGGGGAGGCGACGGCTTCTACGCCATGG |
| | | ACTACTGGGGCCAGGGCACCCTGGTCACCGTGTCTAGCGC |
| | | GTCGACCAAGGGCCCATCGGTCTTCCCCCTGGCACCCTCCT |
| | | CCAAGAGCACCTCTGGGGGCACAGCGGCCCTGGGCTGCCT |
| | | GGTCAAGGACTACTTCCCCGAACCGGTGACGGTGTCGTGG |
| | | AACTCAGGCGCCCTGACCAGCGGCGTGCACACCTTCCCGG |
| | | CTGTCCTACAGTCCTCAGGACTCTACTCCCTCAGCAGCGTG |
| | | GTGACCGTGCCCTCCAGCAGCTTGGGCACCCAGACCTACA |
| | | TCTGCAACGTGAATCACAAGCCCAGCAACACCAAGGTGGA |
| | | CAAGAAAGTTGAGCCCAAATCTTGTGACAAAACTCACACA |
| | | TGCCCACCGTGCCCAGCACCTGAACTCCTGGGGGGACCGT |
| | | CAGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCG |
| | | GGAGGAGCAGTACAACAGCACGTACCGTGTGGTCAGCGTC |
| | | CTCACCGTCCTGCACCAGGACTGGCTGAATGGCAAGGAGT |
| | | ACAAGTGCAAGGTCTCCAACAAAGCCCTCCCAGCCCCCAT |
| | | CGAGAAAACCATCTCCAAAGCCAAAGGGCAGCCCCGAGA |
| | | ACCACAGGTGTACACCCTGCCCCCATCCCGGGAGGAGATG |
| | | ACCAAGAACCAGGTCAGCCTGACCTGCCTGGTCAAAGGCT |
| | | TCTATCCCAGCGACATCGCCGTGGAGTGGGAGAGCAATGG |
| | | GCAGCCGGAGAACAACTACAAGACCACGCCTCCCGTGCTG |
| | | GACTCCGACGGCTCCTTCTTCCTCTATAGCAAGCTCACCGT |
| | | GGACAAGAGCAGGTGGCAGCAGGGGAACGTCTTCTCATGC |
| | | TCCGTGATGCATGAGGCTCTGCACAACCACTACACGCAGA |
| | | AGAGCCTCTCCCTGTCCCCGGGT |
| 19 | Trastuzumab | GACATCCAGATGACCCAGTCCCCCTCCAGCCTGTCCGCCTC |
| | VL | TGTGGGCGACAGAGTGACCATCACCTGTCGGGCCTCCCAG |
| | DNA | GACGTGAACACCGCCGTGGCCTGGTATCAGCAGAAGCCCG |
| | | GCAAGGCCCCCAAGCTGCTGATCTACTCCGCCTCCTTCCTG |
| | | TACTCCGGCGTGCCCTCCCGGTTCTCCGGCTCCAGATCTGG |
| | | CACCGACTTTACCCTGACCATCTCCAGCCTGCAGCCCGAG |
| | | GACTTCGCCACCTACTACTGCCAGCAGCACTACACCACCC |
| | | CCCCCACCTTTGGCCAGGGCACCAAGGTGGAAATCAAG |
| 20 | Trastuzumab | GACATCCAGATGACCCAGTCCCCCTCCAGCCTGTCCGCCTC |
| | light chain | TGTGGGCGACAGAGTGACCATCACCTGTCGGGCCTCCCAG |
| | DNA | GACGTGAACACCGCCGTGGCCTGGTATCAGCAGAAGCCCG |
| | | GCAAGGCCCCCAAGCTGCTGATCTACTCCGCCTCCTTCCTG |
| | | TACTCCGGCGTGCCCTCCCGGTTCTCCGGCTCCAGATCTGG |
| | | CACCGACTTTACCCTGACCATCTCCAGCCTGCAGCCCGAG |
| | | GACTTCGCCACCTACTACTGCCAGCAGCACTACACCACCC |
| | | CCCCCACCTTTGGCCAGGGCACCAAGGTGGAAATCAAGCG |
| | | GACCGTGGCCGCTCCCTCCGTGTTCATCTTCCCACCCTCCG |
| | | ACGAGCAGCTGAAGTCCGGCACCGCCTCCGTCGTGTGCCT |
| | | GCTGAACAACTTCTACCCCCGCGAGGCCAAGGTGCAGTGG |
| | | AAGGTGGACAACGCCCTGCAGTCCGGCAACTCCCAGGAAT |
| | | CCGTCACCGAGCAGGACTCCAAGGACAGCACCTACTCCCT |
| | | GTCCTCCACCCTGACCCTGTCCAAGGCCGACTACGAGAAG |
| | | CACAAGGTGTACGCCTGCGAAGTGACCCACCAGGGCCTGT |
| | | CCAGCCCCGTGACCAAGTCCTTCAACCGGGGCGAGTGC |
| 21 | T(K290C) | GCGTCGACCAAGGGCCCATCGGTCTTCCCCCTGGCACCCT |
| | heavy chain | CCTCCAAGAGCACCTCTGGGGGCACAGCGGCCCTGGGCTG |
| | constant | CCTGGTCAAGGACTACTTCCCCGAACCGGTGACGGTGTCG |
| | region | TGGAACTCAGGCGCCCTGACCAGCGGCGTGCACACCTTCC |
| | DNA | CGGCTGTCCTACAGTCCTCAGGACTCTACTCCCTCAGCAGC |
| | | GTGGTGACCGTGCCCTCCAGCAGCTTGGGCACCCAGACCT |
| | | ACATCTGCAACGTGAATCACAAGCCCAGCAACACCAAGGT |
| | | GGACAAGAAAGTTGAGCCCAAATCTTGTGACAAAACTCAC |
| | | ACATGCCCACCGTGCCCAGCACCTGAACTCCTGGGGGGAC |
| | | CGTCAGTCTTCCTCTTCCCCCCAAAACCCAAGGACACCCTC |
| | | ATGATCTCCCGGACCCCTGAGGTCACATGCGTGGTGGTGG |
| | | ACGTGAGCCACGAAGACCCTGAGGTCAAGTTCAACTGGTA |
| | | CGTGGACGGCGTGGAGGTGCATAATGCCAAGACATGCCCG |
| | | CGGGAGGAGCAGTACAACAGCACGTACCGTGTGGTCAGC |
| | | GTCCTCACCGTCCTGCACCAGGACTGGCTGAATGGCAAGG |
| | | AGTACAAGTGCAAGGTCTCCAACAAAGCCCTCCCAGCCCC |
| | | CATCGAGAAAACCATCTCCAAAGCCAAAGGGCAGCCCCG |
| | | AGAACCACAGGTGTACACCCTGCCCCCATCCCGGGAGGAG |
| | | ATGACCAAGAACCAGGTCAGCCTGACCTGCCTGGTCAAAG |
| | | GCTTCTATCCCAGCGACATCGCCGTGGAGTGGGAGAGCAA |
| | | TGGGCAGCCGGAGAACAACTACAAGACCACGCCTCCCGTG |
| | | CTGGACTCCGACGGCTCCTTCTTCCTCTATAGCAAGCTCAC |
| | | CGTGGACAAGAGCAGGTGGCAGCAGGGGAACGTCTTCTCA |
| | | TGCTCCGTGATGCATGAGGCTCTGCACAACCACTACACGC |
| | | AGAAGAGCCTCTCCCTGTCCCCGGGT |
| 22 | T(K290C) | GAGGTGCAGCTGGTGGAATCCGGCGGAGGCCTGGTCCAGC |
| | heavy chain | CTGGCGGATCTCTGCGGCTGTCTTGCGCCGCCTCCGGCTTC |
| | DNA | AACATCAAGGACACCTACATCCACTGGGTCCGACAGGCAC |
| | | CTGGCAAGGGACTGGAATGGGTGGCCCGGATCTACCCCAC |
| | | CAACGGCTACACCAGATACGCCGACTCCGTGAAGGGCCGG |
| | | TTCACCATCTCCGCCGACACCTCCAAGAACACCGCCTACCT |
| | | GCAGATGAACTCCCTGCGGGCCGAGGACACCGCCGTGTAC |
| | | TACTGCTCCAGATGGGGAGGCGACGGCTTCTACGCCATGG |
| | | ACTACTGGGGCCAGGGCACCCTGGTCACCGTGTCTAGCGC |
| | | GTCGACCAAGGGCCCATCGGTCTTCCCCCTGGCACCCTCCT |
| | | CCAAGAGCACCTCTGGGGGCACAGCGGCCCTGGGCTGCCT |
| | | GGTCAAGGACTACTTCCCCGAACCGGTGACGGTGTCGTGG |
| | | AACTCAGGCGCCCTGACCAGCGGCGTGCACACCTTCCCGG |
| | | CTGTCCTACAGTCCTCAGGACTCTACTCCCTCAGCAGCGTG |
| | | GTGACCGTGCCCTCCAGCAGCTTGGGCACCCAGACCTACA |
| | | TCTGCAACGTGAATCACAAGCCCAGCAACACCAAGGTGGA |
| | | CAAGAAAGTTGAGCCCAAATCTTGTGACAAAACTCACACA |
| | | TGCCCACCGTGCCCAGCACCTGAACTCCTGGGGGGACCGT |
| | | CAGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACATGCCCGCG |
| | | GGAGGAGCAGTACAACAGCACGTACCGTGTGGTCAGCGTC |
| | | CTCACCGTCCTGCACCAGGACTGGCTGAATGGCAAGGAGT |
| | | ACAAGTGCAAGGTCTCCAACAAAGCCCTCCCAGCCCCCAT |
| | | CGAGAAAACCATCTCCAAAGCCAAAGGGCAGCCCCGAGA |
| | | ACCACAGGTGTACACCCTGCCCCCATCCCGGGAGGAGATG |
| | | ACCAAGAACCAGGTCAGCCTGACCTGCCTGGTCAAAGGCT |
| | | TCTATCCCAGCGACATCGCCGTGGAGTGGGAGAGCAATGG |
| | | GCAGCCGGAGAACAACTACAAGACCACGCCTCCCGTGCTG |
| | | GACTCCGACGGCTCCTTCTTCCTCTATAGCAAGCTCACCGT |
| | | GGACAAGAGCAGGTGGCAGCAGGGGAACGTCTTCTCATGC |
| | | TCCGTGATGCATGAGGCTCTGCACAACCACTACACGCAGA |
| | | AGAGCCTCTCCCTGTCCCCGGGT |
| 23 | T(kK183C) | CGGACCGTGGCCGCTCCCTCCGTGTTCATCTTCCCACCCTC |
| | light chain | CGACGAGCAGCTGAAGTCCGGCACCGCCTCCGTCGTGTGC |
| | constant | CTGCTGAACAACTTCTACCCCCGCGAGGCCAAGGTGCAGT |
| | region | GGAAGGTGGACAACGCCCTGCAGTCCGGCAACTCCCAGGA |
| | DNA | ATCCGTCACCGAGCAGGACTCCAAGGACAGCACCTACTCC |
| | | CTGTCCTCCACCCTGACCCTGTCCTGCGCCGACTACGAGAA |
| | | GCACAAGGTGTACGCCTGCGAAGTGACCCACCAGGGCCTG |
| | | TCCAGCCCCGTGACCAAGTCCTTCAACCGGGGCGAGTGC |
| 24 | T(kK183C) | GACATCCAGATGACCCAGTCCCCCTCCAGCCTGTCCGCCTC |
| | light chain | TGTGGGCGACAGAGTGACCATCACCTGTCGGGCCTCCCAG |
| | DNA | GACGTGAACACCGCCGTGGCCTGGTATCAGCAGAAGCCCG |
| | | GCAAGGCCCCCAAGCTGCTGATCTACTCCGCCTCCTTCCTG |
| | | TACTCCGGCGTGCCCTCCCGGTTCTCCGGCTCCAGATCTGG |
| | | CACCGACTTTACCCTGACCATCTCCAGCCTGCAGCCCGAG |
| | | GACTTCGCCACCTACTACTGCCAGCAGCACTACACCACCC |
| | | CCCCCACCTTTGGCCAGGGCACCAAGGTGGAAATCAAGCG |
| | | GACCGTGGCCGCTCCCTCCGTGTTCATCTTCCCACCCTCCG |
| | | ACGAGCAGCTGAAGTCCGGCACCGCCTCCGTCGTGTGCCT |
| | | GCTGAACAACTTCTACCCCCGCGAGGCCAAGGTGCAGTGG |
| | | AAGGTGGACAACGCCCTGCAGTCCGGCAACTCCCAGGAAT |
| | | CCGTCACCGAGCAGGACTCCAAGGACAGCACCTACTCCCT |
| | | GTCCTCCACCCTGACCCTGTCCTGCGCCGACTACGAGAAG |
| | | CACAAGGTGTACGCCTGCGAAGTGACCCACCAGGGCCTGT |
| | | CCAGCCCCGTGACCAAGTCCTTCAACCGGGGCGAGTGC |

In a particular aspect, the invention can be practiced using the anti-HER2 ADCs comprising an antibody designated T(kK183C+K290C), described in U.S. Patent Publication No. 2017/0151341 and International Patent Application Publication WO 2017/093844, each of which is herein incorporated by reference in its entirety. The ani-HER2 antibody T(kK183C+K290C) comprises a heavy chain comprising the amino acid sequence of SEQ ID NO: 14 and a light chain comprising the amino acid sequence of SEQ ID NO:16.

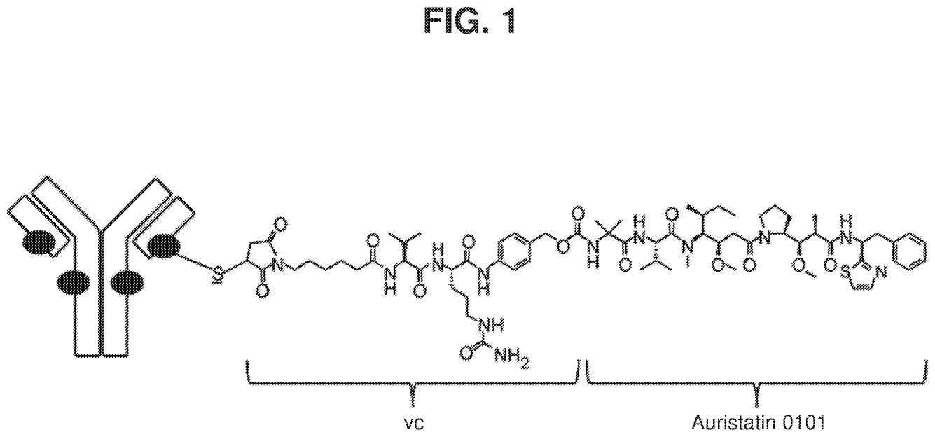

In another aspect, the invention can be practiced using the anti-HER2 ADCs comprises a drug joined to the antibody via a linker, wherein the drug is the auristatin drug 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as 0101)) (Table 2 infra), and the linker is the cleavable linker maleimidocaproyl-valine-citrulline-p-aminobenzyloxycarbonyl (vc) (Table 2 infra). In a particular aspect, the invention can be practiced using the anti-HER2 ADCs T(kK183C+K290C)-vc0101 (see FIG. 1).

TABLE 2
Linker & Payload

| Name | Structure |
|---|---|
| maleimidocaproyl- valine-citrulline-p- aminobenzyloxy- carbonyl (vc) | 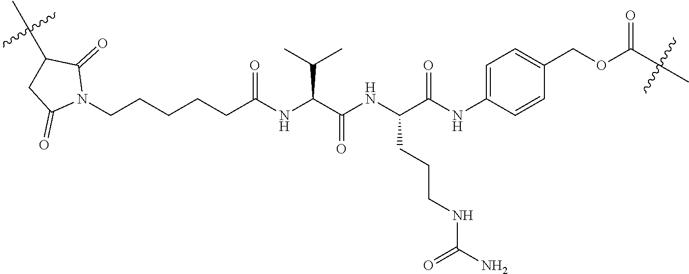 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1] |
| 2-methylalanyl-N- [(3R,4S,5S)- 3-methoxy-1- {(2S)-2-[(1R,2R)- 1-methoxy-2- methyl-3-oxo-3- {[(1S)-2-phenyl- 1-(1,3-thiazol-2- yl)ethyl]amino} propyl]pyrrolidin- 1-yl}-5-methyl- 1-oxoheptan- 4-yl]-N-methyl- L-valinamide (0101) | 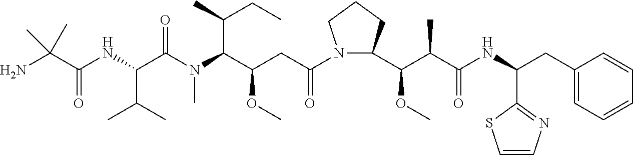 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)C(C)(C)N)C(C)C] |

### HER2− Expressing Cancers

Cancers that may be treated with the dosing regimen or method provided by the present disclosure include HER2 expressing (“HER2 positive” or “HER2+”) solid tumors. the HER2-expressing cancers can express HER2 at a high, moderate, or low level. Methods for identifying levels of expression and/or amplification of the HER2 gene are known in the art, such as immunohistochemistry (IHC) and fluorescence in situ hybridization (FISH or ISH). In some embodiments, the cancers to be treated are breast cancers that are hormone receptor (HR) positive (+). The term “hormone receptor positive” or “HR+” means the tumor is estrogen receptor (ER) positive, progesterone receptor (PR) positive, or both ER positive and PR positive. In some particular embodiments, patients with breast cancer are HR+(including documentation of estrogen receptor (ER) positive and/or progesterone receptor positive tumor ((≥1% positive stained cells) based on most recent tumor biopsy utilizing an assay consistent with local standards) and HER2 IHC+/ISH negative (−) or equivocal. In some other embodiments, the cancer to be treated is resistant to, refractory to and/or relapsed from treatment with trastuzumab and/or trastuzumab emtansine (T-DM1) either of which alone or in combination with a taxane. Examples of cancers to be treated include breast cancer, ovarian cancer, lung cancer, gastric cancer, esophageal cancer, colorectal cancer, urothelial cancer, pancreatic cancer, salivary gland cancer and brain cancer or metastases of the aforementioned cancers. In a more specific embodiment, the breast cancer is hormone receptor positive breast cancer, estrogen receptor and progesterone receptor negative breast cancer, or triple negative breast cancer (TNBC). In another embodiment, the lung cancer is non-small cell lung cancer (NSCLC).

### Pharmaceutical Compositions

Further provided herein are pharmaceutical compositions comprising anti-HER2 ADCs disclosed herein and a pharmaceutically acceptable carrier. The present disclosure also provides articles of manufacture, comprising a container, a composition within the container comprising an anti-HER2 ADC, and a package insert containing instructions to administer a dose of anti-HER2 ADC.

Another aspect of the invention provides for kits containing a formulation comprising a pharmaceutical composition. The kits may comprise an anti-HER2 ADC and a pharmaceutically acceptable carrier. The kits may contain instructions for QW and/or Q3W intravenous dosing of the pharmaceutical composition for the treatment of cancer and/or a HER2-expressing cancer in which the administration of an anti-HER2 ADC is beneficial.

### Combination Therapies

In some aspects of the invention, the dosing regimens or methods described herein further comprises administering to the subject an additional therapeutic agent thereby to elicit additive or potentiated therapeutic effects and/or reduce cytotoxicity of some anti-cancer agents. Examples of the additional therapeutic agents include chemotherapy, radiation, surgery, hormone therapy, therapeutic antibodies, ADCs, immunomodulating agents, cytotoxic agents, and cytostatic agents. A cytotoxic effect refers to the depletion, elimination and/or the killing of a target cells (i.e., tumor cells). A cytotoxic agent refers to an agent that has a cytotoxic and/or cytostatic effect on a cell. A cytostatic effect refers to the inhibition of cell proliferation. A cytostatic agent refers to an agent that has a cytostatic effect on a cell, thereby inhibiting the growth and/or expansion of a specific subset of cells (i.e., tumor cells). An immunomodulating agent refers to an agent that stimulates the immune response though the production of cytokines and/or antibodies and/or modulating T cell function thereby inhibiting or reducing the growth of a subset of cells (i.e., tumor cells) either directly or indirectly by allowing another agent to be more efficacious. The anti-HER2 ADCs may be co-formulated with the additional therapeutic agents or formulated separately with the additional therapeutic agents.

The anti-HER2 ADC and/or one or more additional therapeutic agents may be administered within any time frame suitable for performance of the intended therapy. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s).

The disclosed combination therapies may elicit a synergistic therapeutic effect, i.e., an effect greater than the sum of their individual effects or therapeutic outcomes. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

Examples of specific combination therapies encompassed by this invention are set forth in Examples 1 and 2 hereinbelow.

### EXAMPLES

The following examples are meant to illustrate the methods and materials of the present invention. Suitable modifications and adaptations of the described conditions and parameters normally encountered in the art that are obvious to those skilled in the art are within the spirit and scope of the present invention.

### Example 1

### Anti-HER2 T(kK183C+K290C)-vc0101 ADC Clinical Study

A. Study Overview

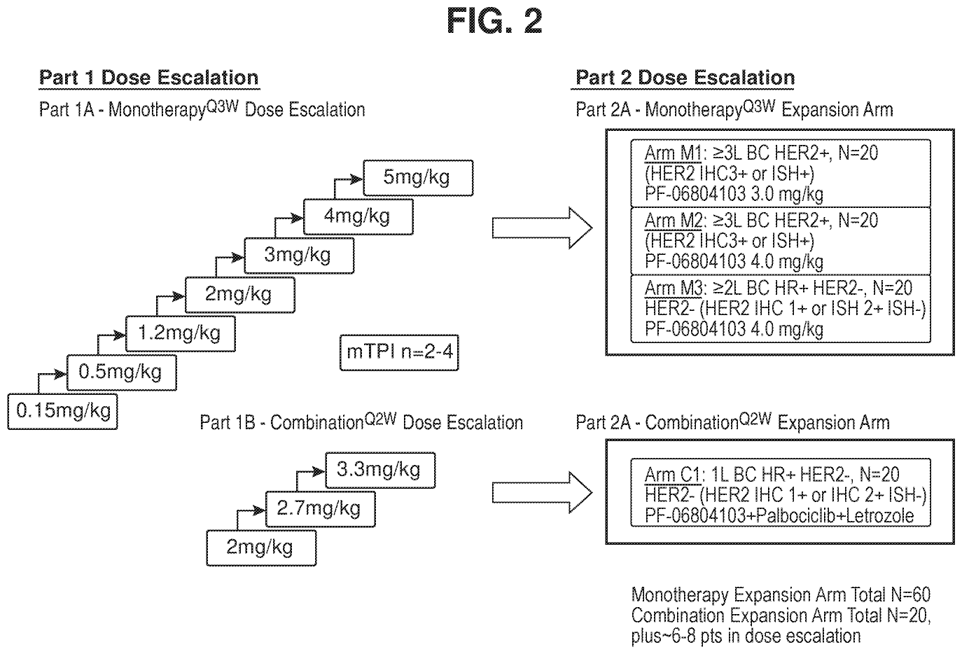

This example illustrates a Phase 1, open-label, multicenter, multiple dose, safety, PK, and PD study of single-agent T(kK183C+K290C)-vc0101 ADC (PF-06804103), in sequential cohorts (n=2-15) of adult patients with HER2+solid tumors (breast cancer (BC) and gastric cancer (GC)) and in postmenopausal patients with HR+HER2 IHC 1+or IHC 2+/ISH− breast cancer (BC), and resistant or intolerant to standard therapy or for which no standard therapy is available, received increasing doses of single-agent T(kK183C+K290C)-vc0101 ADC administered intravenously every 21 days. This study contains two parts, dose escalation (Part 1) and dose expansion (Part 2). Part 1A and 1B evaluated escalating doses of T(kK183C+K290C)-vc0101 ADC as monotherapy and as part of a combination regimen, respectively. Part 2A and Part 2B will evaluate selected doses of T(kK183C+K290C)-vc0101 ADC in expansion cohorts as monotherapy and in a combination regimen, respectively. The overall study design is described in FIG. 2.

In Part 1A, patients with HER2− positive BC or HER2− positive GC received escalating doses of T(kK183C+K290C)-vc0101 ADC starting at 0.15 mg/kg, Q3W in a 21-day cycle to estimate the dose level of T(kK183C+K290C)-vc0101 ADC to be administered in Part 2A.

In Part 1B, postmenopausal patients with HR-positive HER2 IHC 1+ or IHC 2+/ISH− BC will receive escalating doses of T(kK183C+K290C)-vc0101 ADC starting at the dose equivalent to the recommended monotherapy Q3W Part 2 dose minus 1 dose, Q2W in a 28-day cycle, administered in combination with SOC doses of palbociclib and letrozole (as per local and regional guidelines). Data collected during Part 1B informed the dose levels selected for dose expansion in Part 2B.

In Part 2A, HER2-positive BC patients in 3L setting will be randomly assigned to receive 3 mg/kg or 4 mg/kg doses of T(kK183C+K290C)-vc0101 ADC administered as monotherapy Q3W to further evaluate safety, efficacy, and to evaluate the benefit/risk of 3 mg/kg and 4 mg/kg Q3W in a larger population to support optimal dose selection. Also in Part 2A, HR-positive HER2 IHC1+or IHC 2+/ISH− BC patients in 2L setting will receive 4 mg/kg of T(kK183C+K290C)-vc0101 ADC administered as monotherapy Q3W. A lower dose (eg., 3 mg/kg) will be tested if the observed toxicity of 4 mg/kg Q3W is determined to be too high.

In Part 2B, patients with HR-positive HER2 IHC 1+or IHC 2+/ISH− BC in the 1L setting will receive the selected T(kK183C+K290C)-vc0101 ADC dose administered Q2W (Part 1B) in a 28-day cycle in combination with SOC doses of palbociclib and letrozole (as per local and regional guidelines).

Treatment with T(kK183C+K290C)-vc0101 ADC continued until either disease progression, patient refusal, or unacceptable toxicity occurred, unless the investigator and medical monitor agreed to treatment beyond progression based on individual benefit/risk assessments.

In both study parts, the proposed dose levels, schedules, and PK time points could be reconsidered based on emerging safety and PK data. A dose level or treatment arm could be discontinued at any time depending on the totality of the data including, but not limited to the evaluation of all available clinical, safety, PK, PD, and preliminary efficacy results.

Primary objectives were to evaluate the safety and tolerability of T(kK183C+K290C)-vc0101 ADC, characterize its dose-limiting toxicities (DLTs), and determine the recommended Phase 2 dose (RP2D) in adult patients with Her2+ cancer of the breast (BC) or the stomach and esophagogastric junction (GC). A modified Toxicity Probability Interval design targeting a DLT rate of approximately 27.5% with an equivalence interval of 22.5%, 32.5% was used in the dose escalation phase of the study. Secondary objectives were to evaluate PK characteristics, immunogenicity, and preliminary anti-tumor activity of T(kK183C+K290C)-vc0101 ADC. Assessment of response was made using Response Evaluation Criteria in Solid Tumors v1.1 (RECIST v1.1). Objective response rate is calculated for response-evaluable patients, i.e., patients with a target lesion at baseline and ≥1 post-baseline assessment up to the time of progressive disease or new anti-cancer therapy.

B. Patient Population

All patients being considered for the study and eligible for screening were required to sign an informed consent for the study before completing any study-specific procedures.

Key inclusion criteria for Part 1 included: adult patient (age≥18 years) with histological or cytological diagnosis of advanced/unresectable or metastatic HER2 positive BC or metastatic HER2 positive adenocarcinoma of the stomach or esophagogastric junction (GC) that is refractory to or intolerable with standard therapy or for which no standard therapy is available. HER2 positivity is defined according to the American Society of Clinical Oncology/College of American Pathologists Guidelines. Documentation of HER2 gene amplification or overexpression by one of the following is required:

Overexpression by immunohistochemistry (IHC) categorized as HER2 3+ defined as:
Breast Cancer: circumferential membrane staining that is complete, intense and in >10% of tumor cells.
Gastric Cancer (Part 1A only): Surgical specimen: strong, complete/basolateral or lateral membranous reactivity in ≥10% of cells.
Gastric Cancer (Part 1A only): Biopsy specimen: tumor cell cluster (>5 tumor cells) with strong, complete basolateral or lateral membranous activity irrespective of percentage of tumor cells stained.

Overexpression by IHC categorized as HER2 2+ defined as:
Breast Cancer: weak to moderate complete membrane staining observed in >10% of tumor cells (in situ hybridization (ISH) confirmation if IHC is equivocal).
Gastric Cancer (Part 1A only): Surgical specimen: weak to moderate complete basolateral or lateral membranous reactivity in ≥10% of cells (ISH confirmation if IHC is equivocal). Gastric Cancer (Part 1A only): Biopsy specimen: tumor cell cluster with weak to moderate, complete basolateral or lateral membranous activity irrespective of percentage of tumor cells stained (ISH confirmation if IHC is equivocal).

Overexpession by IHC categorized as HER2 1+ defined as:
BC: incomplete membrane staining that is faint/barely perceptible and in >10% of tumor cells.
GC (Part 1A only): surgical specimen: faint/barely perceptible membranous reactivity in ≥10% of tumor cells; cells reactive only in part of their membrane.
GC (Part 1A only): biopsy specimen: tumor cell cluster with faint or barely membranous reactivity irrespective of tumor cells stained.

Overexpession by IHC categorized as HER2 0 defined as:
BC: no staining is observed OR membrane staining that is incomplete and is faint/barely perceptible in ≤10% of tumor cells.
GC (Part 1A only): surgical specimen—no reactivity to membranous reactivity in ≤10% of tumor cells.
GC (Part 1A only): biopsy specimen—no reactivity in any tumor cells.

Gene amplification by ISH defined as:
Single-probe: average HER2 copy number ≥6.0 signals/cell; OR
Single-probe: average HER2 copy number ≥4.0 and <6.0 signals/cell and Concurrent IHC 3+ and/or concurrent dual-probe ISH Group 1.
Dual-probe: HER2/chromosome enumeration probe 17 (CEP17) with a ratio >2.0 with an average HER2 copy number ≥4.0 signals/cell (Group 1).
<4.0 signals/cell (Group 2) and IHC 3+.
Dual-probe HER2/CEP17 ratio <2.0.
Average HER2 copy number ≥6.0 signals/cell (Group 3) requires additional work-up (IHC 3+, or IHC2+ and recount of ISH with observer blinded to previous results, counting at least 20 cells, shows a HER2/CEP17 Ratio <2.0 and an average HER2 signals/cell >6.0).
Average HER2 copy number >4.0 and 56.0 signals/cell (Group 4) and IHC 3+.

Previous HER2 positive test results, using a Food and Drug Administration (FDA) approved or locally validated test will be accepted.

More specifically, patient inclusion criteria include, but is not limited to, the following: 1) Parts 1A & 2A (Arms M1 and M2)

a) patients age ≥18 years;

b) advanced/unresectable or metastatic HER2-positive BC or metastatic HER2 positive adenocarcinoma of the stomach or esophagogastric junction that is refractory to or intolerable with standard therapy or for which no standard therapy is available; and

c) documented histologically or cytologically confirmed diagnosis of HER2 positive BC or metastatic HER2-positive adenocarcinoma of the stomach or esophagogastric junction based on local laboratory results; 2) Part 2A (Arm M3)

a) adult female patients age ≥18 years;

b) advanced/unresectable or metastatic HER2 IHC 1+ or IHC 2+/ISH− BC that has progressed on at least 1 prior line of systemic therapy including a hormonal based regimen; and

c) documented HER2 IHC 1+ or IHC 2+/ISH− BC histologically or cytologically defined as either HER2 IHC 1+ or IHC 2+/ISH− based on local laboratory results. Documentation of HER2 IHC and/or ISH status; and 3) Parts 1B and 2B

a) adult female patients age ≥18 years;

b) postmenopausal women, defined as: (i) prior bilateral surgical oophorectomy, or medically confirmed postmenopausal status defined as spontaneous cessation of regular menses for at least 12 consecutive months or FSH and estradiol blood levels in their respective postmenopausal ranges with no alternative pathological or physiological cause;

c) advanced/unresectable or metastatic HER2 IHC 1+ or IHC 2+/ISH+ BC previously untreated with any systemic anti-cancer therapy; and

d) documentation of histologically or cytologically confirmed diagnosis of HER2 IHC1+ or IHC 2+/ISH− BC based on local laboratory results. Documentation of HER2 IHC and/or ISH status.

Patients were excluded from this study if they met the following key exclusion criteria:
a) patients with Her2 IHC 0 defined as:
   (i) BC: no staining is observed OR membrane staining that is incomplete and is faint/barely perceptible in ≤10% of tumor cells;
   (ii) GC (Part 1A only): surgical specimen-no reactivity to membranous reactivity in ≤10% of tumor cells; or biopsy specimen-no reactivity in any tumor cells; and
b) patients with known symptomatic brain metastases requiring steroid treatment; and
c) patients having major surgery or systemic anticancer therapy within 4 weeks of starting treatment.

C. Treatment Schedule

Part 1A—Monotherapy Dose Escalation with T(kK183C+K290C)-vc0101 ADC

The objective was to evaluate the safety, tolerability, and antitumor activity of PF-06804103, characterize its dose-limiting toxicity (DLT) and determine the recommended phase 2 dose in adult patients with HER2+ cancer of the breast (BC), and the stomach and esophagogastric junction (GC) in the dose escalation part of a phase 1 study.

In the dose escalation part (Part 1) T(kK183C+K290C)-vc0101 ADC (PF-06804103) was administered as an intravenous (IV) infusion every 21 days (Q3W) with a starting dose of 0.15 mg/kg. Based on clinical and PK data, an alternate dosing schedule could be evaluated. Treatment with T(kK183C+K290C)-vc0101 ADC continued until either disease progression, patient refusal/withdrawal of consent, or unacceptable toxicity occurred, whichever occurred first, unless the investigator and medical monitor agreed to treatment beyond progression based on individual benefit/risk assessments.

A modified toxicity probability interval (mTPI) method targeting a DLT rate of approximately 27.5% with an equivalence interval of (22.5%, 32.5%) was utilized in Part 1 of the study.

The dose levels planned for Part 1 of the study are shown in Table 3. Intermediate doses could be explored, if appropriate based on emerging safety, PK or PD data.

TABLE 3
T(kK183C+K290C)-vc0101 ADC Dose Escalation Levels

| | T(kK183C+K290C)- |
|---|---|
| | vc0101 ADC Dose |
| Dose Level | (mg/kg) |
| 1 (Starting Dose) | 0.15 |
| 2 | 0.5 |
| 3 | 1.2 |
| 4 | 2.0 |
| 5 | 3.0 |
| 6 | 4.0 |
| 7 | 5.0 |
| 8 | 6.0 |
| 9 | n |

### Assessments

Safety assessments included collection of AEs, SAES, vital signs and physical examination, ECG (12 lead), ECHO or MUGA, diffusing capacity of the lungs for carbon dioxide (DLco), ophthalmic examination, laboratory safety assessments, including pregnancy tests and verification of concurrent medications.

Pharmacokinetic assessments included quantifying the serum concentrations of T(kK183C+K290C)-vc0101 ADC (measured as conjugated payload), total antibody, and unconjugated payload using validated bioanalytical assays on blood samples collected before treatment and during the study. Specifically, total antibody concentrations were measured using ELISA method, T(kK183C+K290C)-vc0101 ADC concentrations were measured as conjugated payload using a hybrid LC-MS/MS method, and unconjugated payload concentrations will be measured using an LC-MS/MS method. For preliminary PK assessment, mean serum concentration-time profiles of T(kK183C+K290C)-vc0101 ADC were generated for each dose cohort; Noncompartmental PK parameters were estimated from Cycle 1 concentration-time data using nominal sampling time. For T(kK183C+K290C)-vc0101 ADC and total antibody, PK parameters including the maximum plasma concentration (Cmax), time to maximum plasma concentration (Tmax), and area under the plasma concentration versus time curve (AUC_{inf}, AUC_{τ}), clearance (CL), volume of distribution at steady state (Vss), terminal half-life (t_{1/2}), and accumulation ratio (R_{ac}) were calculated. For unconjugated payload, PK parameters including Cmax, Tmax, AUCi_{nf}, AUCτ, t_{1/2}, and R_{ac}, were calculated.

Antitumor clinical activity was assessed using computed tomography or magnetic resonance imaging at baseline and then every 6 weeks after the start of treatment until confirmed progressive disease or discontinuation of study treatment. After 6 months of study treatment, assessments could be performed every 12 weeks.

Tumor response was assessed according to RECIST v1.1. The objective response rate (ORR) was calculated for response-evaluable patients with tumor assessment at baseline and ≥1 determinate post-baseline assessment (including unconfirmed responses). Changes in tumor size was categorized as complete response (CR), partial response (PR), stable disease (SD), or progressive disease (PD), the latter incorporating the appearance of new lesions, as defined hereinbelow:

i) Complete Response (CR): Complete disappearance of all target lesions with the exception of nodal disease. All target nodes must decrease to normal size (short axis <10 mm). All target lesions must be assessed;

ii) Partial Response (PR): Greater than or equal to 30% decrease under baseline of the sum of diameters of all target measurable lesions. The short diameter is used in the sum for target nodes, while the longest diameter is used in the sum for all other target lesions. All target lesions must be assessed;

iii) Stable: Does not qualify for CR, PR or Progression. All target lesions must be assessed. Stable can follow PR only in the rare case that the sum increases by less than 20% from the nadir, but enough that a previously documented 30% decrease no longer holds; and

iv) Objective Progressive Disease (PD): 20% increase in the sum of diameters of target measurable lesions above the smallest sum observed (over baseline if no decrease in the sum is observed during therapy), with a minimum absolute increase of 5 mm.

### Results

1. Patients

Key Inclusion Criteria included, but were not limited to:
a) Histological or cytological diagnosis of advanced/unresectable or metastatic HER2+ BC or metastatic HER2+ GC refractory to standard therapy or for which no standard therapy is available;
b) Eastern Cooperative Oncology Group performance status (ECOG PS) <1; and
c) Adequate bone marrow, renal, and hepatic function.

16 (n=6 BC and n=10 GC) of 35 (46%) patients provided a total of 18 tumor samples. Based on HER2 immunohistochemistry (IHC) testing, 12 patients had scores of 3+ and 4 patients had scores of 2+. Patients who scored 2+ were all tested as FISH+. The median (range) number of prior treatments received was 3 (1-7) and 6 (3-18) for GC and BC patients, respectively (Table 4). All patients had received prior HER2-targeted therapy; all patients with GC and BC had received trastuzumab (Table 4).

TABLE 4
Prior Cancer Treatments

| | Gastric/ | | |
|---|---|---|---|
| | Esophageal | Breast | |
| | Cancer | Cancer | Total |
| n (%) | n = 15 | n = 20 | N = 35 |
| Received prior | 15 (100.0) | 20 (100.0) | 35 (100.0) |
| cancer treatment | | | |

Number of prior regimens

| | | | |
|---|---|---|---|
| 1-3 | 8 (53.3) | 1 (5) | 9 (25.7) |
| 4-6 | 6 (40.0) | 11 (55.0) | 17 (48.6) |
| >6 | 1 (6.7) | 8 (40.0) | 9 (25.7) |
| number of prior | 3 (1-7) | 6 (3-18) | 5 (118) |
| treatments, | | | |
| median (range) | | | |
| Pertuzumab | 0 (0.0) | 14 (70.0) | 14 (40.0) |
| Trastuzumab | 15 (100.0) | 20 (100.0) | 34 (97.1) |
| T-DMI | 0 (0.0) | 17 (85.0) | 17 (48.6) |

T-DM1 = trastuzumab emtansine

TABLE 5
Patient Demographics and Baseline Characteristics (Safety Analysis Set)

| PF-06804103 Dose | | | | | | | |
|---|---|---|---|---|---|---|---|
| (mg/kg) | | | | | | | |
| 0.15 | 0.5 | 1.2 | 2.0 | 3.0 | 4.0 | 5.0 | Total |
| n = 2 | n = 2 | n = 2 | n = 4 | n = 10 | n = 9 | n = 6 | N = 35 |

Sex, n (%)

| | | | | | | | | | | | | | | | | |
|---|---|---|---|---|---|---|---|---|---|---|---|---|---|---|---|---|
| Male | 2 | (100.0) | 1 | (50.0) | 1 | (50.0) | 2 | (50.0) | 4 | (40.0) | 1 | (11.1) | 2 | (33.3) | 13 | (37.1) |
| Female | 0 | (0.0) | 1 | (50.0) | 1 | (50.0) | 2 | (50.0) | 6 | (60.0) | 8 | (88.9) | 4 | (66.7) | 22 | (62.9) |

Race, n (%)

| | | | | | | | | | | | | | | | | |
|---|---|---|---|---|---|---|---|---|---|---|---|---|---|---|---|---|
| White | 1 | (50.0) | 1 | (50.0) | 2 | (100.0) | 2 | (50.0) | 8 | (80.0) | 6 | (66.7) | 5 | (83.3) | 25 | (71.4) |
| Black | 1 | (50.0) | 0 | (0.0) | 0 | (0.0) | 0 | (0.0) | 0 | (0.0) | 0 | (0.0) | 0 | (0.0) | 1 | (2.9) |
| Asian | 0 | (0.0) | 0 | (0.0) | 0 | (0.0) | 2 | (50.0) | 2 | (20.0) | 3 | (33.3) | 1 | (16.7) | 8 | (22.9) |
| Not | 0 | (0.0) | 1 | (50.0) | 0 | (0.0) | 0 | (0.0) | 0 | (0.0) | 0 | (0.0) | 0 | (0.0) | 1 | (2.9) |
| reported | | | | | | | | | | | | | | | | |
| Age, | 49.0 | (35-63) | 65.5 | (65-66) | 58.0 | (50-66) | 65.0 | (64-74) | 53.5 | (36-70) | 53.0 | (41-65) | 58.5 | (39-71) | 58.0 | (35-74) |
| median | | | | | | | | | | | | | | | | |
| (range), | | | | | | | | | | | | | | | | |
| years | | | | | | | | | | | | | | | | |

ECOG PS*, n (%)

| | | | | | | | | | | | | | | | | |
|---|---|---|---|---|---|---|---|---|---|---|---|---|---|---|---|---|
| 0 | 0 | (0.0) | 0 | (0.0) | 2 | (100) | 0 | (0.0) | 4 | (40.0) | 2 | (22.2) | 4 | (66.7) | 12 | (34.3) |
| 1 | 2 | (100) | 2 | (100) | 0 | (0.0) | 4 | (100.0) | 6 | (60.0) | 7 | (77.8) | 2 | (33.3) | 23 | (65.7) |

Primary diagnosis

| Gastric | 2 | 2 | 1 | 2 | 5 | 1 | 2 | 15 |
|---|---|---|---|---|---|---|---|---|
| Cancer | | | | | | | | |
| (GC) | | | | | | | | |
| Breast | 0 | 0 | 1 | 2 | 5 | 8 | 4 | 20 |
| Cancer | | | | | | | | |
| (BC) | | | | | | | | |

*ECOG PS = Eastern Cooperative Oncology Group performance status

2. Clinical Activity

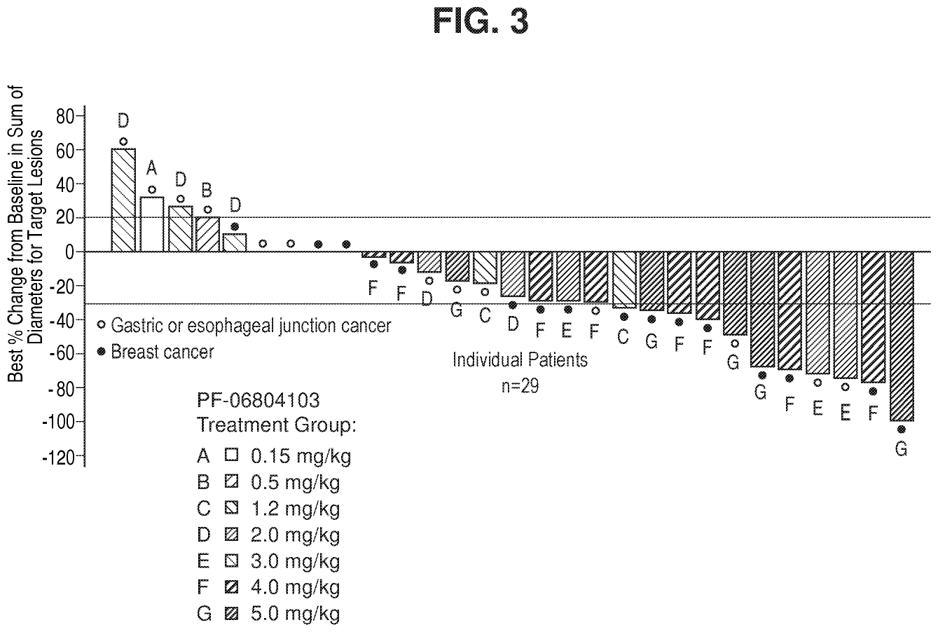

Treatment with PF-06804103 resulted in an objective response rate (ORR) of 38.7% based on all response-evaluable patients across all doses (Table 6). For patients who received ≥3 mg/kg PF-06804103: (i) ORR was 11/21 (52.4%); 8/21 (38.1%) patients achieved stable disease; and (ii) complete response was observed in 2/21 (9.5%) patients receiving PF-06804103 (Table 6). Median duration of response was 6.9 months in patients with confirmed or unconfirmed responses. The best change in tumor size in patients with BC or GC is shown in FIG. 3.

TABLE 6
Summary of Tumor Assessments in Response-Evaluable Patients
PF-06804103 Dose (mg/kg)

| | | | | | |
|---|---|---|---|---|---|
| <2.0 | 2.0 | 3.0 | 4.0 | 5.0 | Total |
| n = 6 | n = 4 | n = 8 | n = 8 | n = 5 | N = 31 |

Best overall response, n (%)*

| | | | | | | | | | | | | |
|---|---|---|---|---|---|---|---|---|---|---|---|---|
| CR | 0 | (0.0) | 0 | (0.0) | 0 | (0.0) | 1 | (12.5) | 1 | (20.0) | 2 | (6.5) |
| PR | 1 | (16.7) | 0 | (0.0) | 2 | (25.0) | 4 | (50.0) | 3 | (60.0) | 10 | (32.3) |
| SD | 3 | (50.0) | 4 | (100.0) | 4 | (50.0) | 3 | (37.5) | 1 | (20.0) | 15 | (48.4) |
| PD | 2 | (33.3) | 0 | (0.0) | 2 | (25.0) | 0 | (0.0) | 0 | (0.0) | 4 | (12.9) |

| | | | | | | |
|---|---|---|---|---|---|---|
| ORR, % | 16.7 | 0 | 25.0 | 62.5 | 80.0 | 38.7 |

*Includes confirmed and unconfirmed responses.
CR = complete response; ORR = objective response rate; PD = progressive disease; PR = partial response; SD = stable disease

3. PK Characterization of PF-06804103

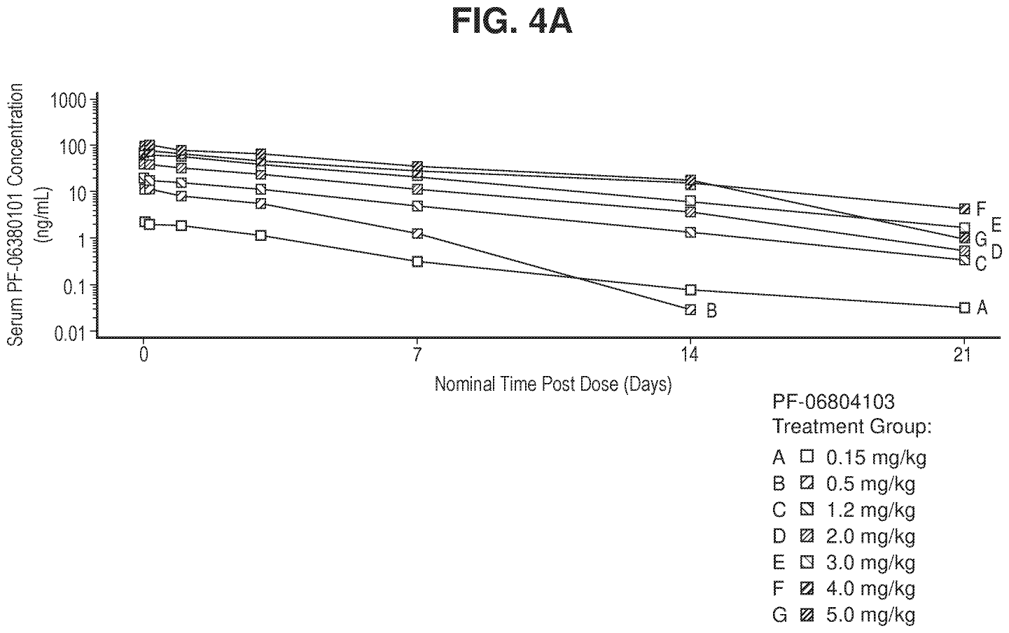

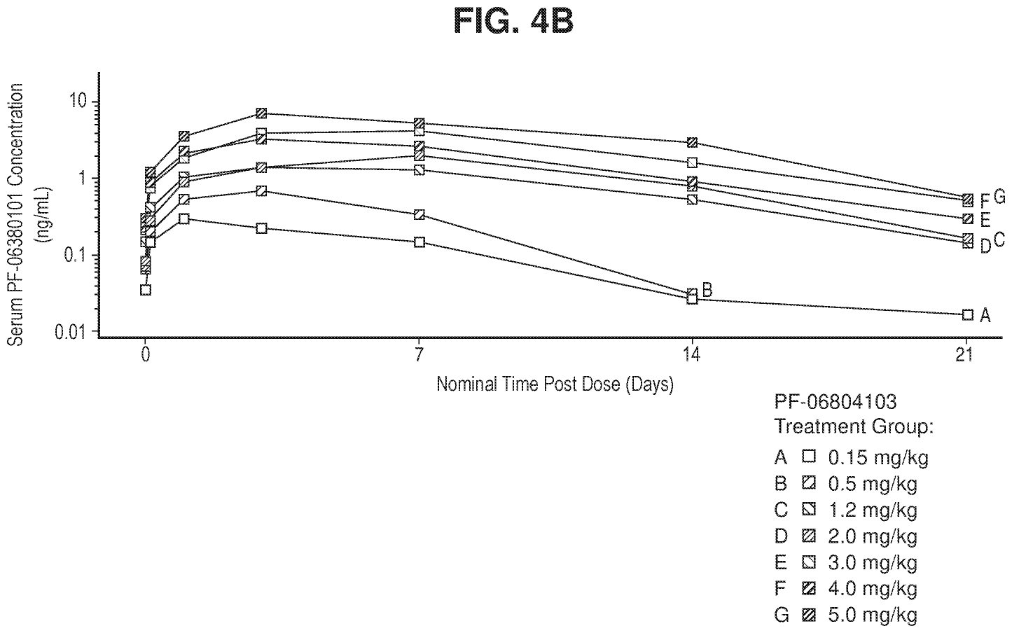

Dose-dependent increases in the exposure of the ADC and the unconjugated payload were observed following IV administration of PF-06804103 (FIGS. 4A & 4B).

Serum concentrations of the unconjugated payload were substantially lower than those of ADC (FIGS. 4A & 4B); and the half-life of ADC ranged from 2 to 5 days (Table 7).

TABLE 7
PK Parameters for PF-06804103 ADC, by dose (mg/kg)

| Mean | 0.15 | | 0.5 | | 1.2 | | 2.0 | | 3.0 | | 4.0 | | 5.0 | |
|---|---|---|---|---|---|---|---|---|---|---|---|---|---|---|
| (% CV) | n = 2 | | n = 2 | | n = 2 | | n = 4 | | n = 10 | | n = 9 | | n = 6 | |
| AUC_{inf} | 12.1 | (—) | 35.2 | (21) | 96.1 | (13) | 220 | (12) | 359 | (27) | 445 | (23) | 723 | (37) |
| (μg · day/mL) | | | | | | | | | | | | | | |
| Cₘₐₓ (μg/mL) | 2.29 | (20) | 11.3 | (17) | 19.2 | (21) | 37.8 | (23) | 69.3 | (23) | 81.3 | (21) | 106 | (27) |
| CL (L/day) | 0.991 | (—) | 1.36 | (12) | 0.787 | (19) | 0.502 | (32) | 0.523 | (31) | 0.499 | (25) | 0.535 | (42) |
| V_{z} (L) | 5.32 | (—) | 3.77 | (24) | 4.13 | (22) | 2.94 | (30) | 2.98 | (17) | 3.25 | (17) | 3.64 | (26) |
| T_{1/2} (day) | 3.73 | (—) | 1.92 | (12) | 3.63 | (3) | 4.14 | (18) | 4.24 | (30) | 4.59 | (12) | 4.93 | (13) |

% CV = percent coefficient variation; AUC = area under the plasma concentration-time curve from time 0 to infinity; CL = clearance; Cₘₐₓ = maximum plasma concentration; PK = pharmacokinetic

4. Safety

The most common treatment-related adverse events (AEs) (any grade) were alopecia and fatigue. Grade 3-4 treatment-related AEs reported included fatigue, peripheral neuropathy, myalgia, arthralgia, and decreased appetite. 5 (14.3%) patients reported grade 3-4 treatment-related AEs in the first cycle of treatment.

Dose-limiting toxicities (DLTs) (mostly grade 3) were reported in 3 patients and included arthralgia, neuropathy, myalgia, fatigue, and osteomuscular pain.

The proportion of patients with AEs that led to dose reduction, interruption, or drug withdrawal (by PF-06804103 dose group) was 100% (0.15 mg/kg), 0 (0.5 mg/kg), 50% (1.2 mg/kg), 25% (2.0 mg/kg), 40% (3.0 mg/kg), 78% (4.0 mg/kg) and 83% (5.0 mg/kg).

5. Conclusion

In this small group of heavily pretreated GC and BC patients, treatment with the PF- 06804103 ADC showed promising efficacy and a generally manageable toxicity profile. ORR was 52.4% among response-evaluable patients who received ≥3 mg/kg PF-06804103, which included 2 (9.5%) complete responses.

### Part 1B—Combination Regimen Dose Escalation

The combination regimen evaluated in Part 1B will be administered to patients with 1L BC HR-positive HER2 IHC 1+ or IHC 2+/ISH−.

PF-06804103 will be administered by IV infusion every 14 days in combination with SOC oral palbociclib and oral letrozole. Dose escalation up to 3.3 mg/kg Q2W or de-escalation (Table 8) including higher, intermediate, or lower doses may be evaluated based on all available clinical, safety, PK, and/or PD data.

The starting dose level of PF-06804103 is planned to be at the equivalent to monotherapy Part 2 dose minus 1 and was selected based on potential DDI, any overlapping toxicity considerations, and all available clinical, safety, PK, tolerability, and preliminary efficacy data.

TABLE 8
Part 1B-PF-06804103 Dose Escalation Levels

| | PF-06804103 Dose |
|---|---|
| Dose Level | (mg/kg) Q2W* |
| −1 | 1.3 |
| 1 (starting dose) | 2.0 |
| 2 | 2.7 |
| 3 | 3.3 |

*Intermediate, or lower doses may be explored

Palbociclib is a weak time-dependent inhibitor of CYP3A and is expected to cause a low to moderate increase in exposure for unconjugated payload PF-06804103. Since the monotherapy Part 2 dose minus 1 is 3 mg/kg Q3W, the starting dose of PF-06804103 in Part 1B will be 2 mg/kg Q2W, to yield the same dosing intensity ad 3 mg/kg Q3W in monotherapy dosing. The expected maximum Part 1B dose will be 2.7 mg/kg Q2W, to yield the same dosing intensity as 4 mg/kg Q3W monotherapy dosing. Higher doses of PF-06804103 may be tolerated by patients previously untreated with systemic anticancer therapies. For those patients, the maximum Part 1B dose may exceed 2.7 mg/kg QW.

More specifically, PF-06804103 will be administered IV at a starting dose of 2 mg/kg Q2W+palbociclib (125 mg)+letrozole (2.5 mg) Q4W.

### Part 2A—Monotherapy Dose Expansion

In Part 2A, HER2-positive BC patients in 3L setting will be randomly assigned to receive 3 mg/kg or 4 mg/kg doses of T(kK183C+K290C)-vc0101 ADC administered as monotherapy Q3W to further evaluate safety, efficacy, and to evaluate the benefit/risk of 3 mg/kg and 4 mg/kg Q3W in a larger population to support optimal dose selection. Also in Part 2A, HR-positive HER2 IHC1+ or IHC 2+/ISH− BC patients in 2L setting will receive 4 mg/kg of T(kK183C+K290C)-vc0101 ADC administered as monotherapy Q3W. A lower dose (eg., 3 mg/kg) will be tested if the observed toxicity of 4 mg/kg Q3W is determined to be too high.

Dose levels of PF-06804103 to be administered will be selected following a review of all available safety, tolerability, preliminary efficacy, and PK data collected in Part 1A. The planned Part 2 monotherapy dose for PF-06804103 are 3.0 mg/kg/ and 4.0 mg/kg Q3W. More specifically, study treatments in Part 2A include the following:
Arm M1: PF-06804103 will be administered IV at 3 mg/kg Q3W;
Arm M2: PF-06804103 will be administered IV at 4 mg/kg Q3W; and
Arm M3: PF-06804103 will be administered IV at 4 mg/kg Q3W.

### Part 2B—Combination Dose Regimen Expansion

In Part 2B, patients with HR-positive HER2 IHC 1+ or IHC 2+/ISH− BC in the 1L setting received the selected T(kK183C+K290C)-vc0101 ADC dose administered Q2W (Part 1B) in a 28-day cycle in combination with SOC doses of palbociclib and letrozole (as per local and regional guidelines).The SOC administration of palbociclib is in 28-day cycles, the dose level selection of PF-06804103 Q2W will be based on all available clinical, safety, tolerability, preliminary efficacy, and PK data from Part 1B. The anticipated Part 2 combination dose for PF-06804103 is 2.7 mg/kg Q2W.

More specifically, study treatments in Part 2B include the following:

Arm Cl: PF-06804103 (TBD) Q2W will be administered IV+palbociclib (125 mg)+letrozole (2.5 mg) Q4W (Table 9).

TABLE 9
Dose Levels for Part 2B

| | Regimen | | |
|---|---|---|---|
| Arm | PF-06804103 | Palbociclib¹ | Letrozole |
| Cl | Dose TBD | 125 mg QD for 3 | 2.5 mg PO |
| | mg/kg IV | weeks¹ repeat | QD on Days 1 |
| | Q2W² | every 28 days | through 28 |

¹3 weeks on followed by 1 week off
²PF-06804103 dose level to be administered in combination with palbociclib ad letrozole to be established from Part 1B.

### Example 2

### Anti-HER2 T(kK183C+K290C)-vc0101 ADC Part 2 Study (Alternate): Combination Dose Finding (Part 2A) and Dose Expansion as a Single Agent (Part 2B: Arm A, B, C and D) and in Combination (Part 2B: Arms 1, 2 and 3)

A. Overview:

Part 2 may also further evaluate the dose selected from Part 1 as a single agent and in combination in patients with:

Single Agent:

Arm A: HER2+BC (HER2 IHC3+ or IHC2+ISH+ (in situ hybridization) BC;

Arm B: Hormone receptor (HR)+ HER2 IHC2+ ISH− or equivocal BC;

Arm C: HER2+ (HER2 IHC3+ or IHC2+ ISH+) GC or HER2 IHC2+ ISH− or equivocal GC; and

Arm D: NSCLC (all corners); and

Combination:

Arm 1 and Arm 2: “First Line (1L) MBC”: HER2+; and

Arm 3: “First Line (1L) MBC”: HR+ HER2− mBC, with either failure of adjuvant treatment, or de novo MBC; with no prior exposure to CDK4/6 inhibitors.

The single agent T(kK183C+K290C)-vc0101 ADC MTD/RP2D from Part 1 will be used to initiate the Part 2 single agent dose expansion arm studies (Arm A, B, C and D). Additionally, the starting dose of T(kK183C+K290C)-vc0101 ADC in combination studies will be based on the MTD/RP2D from Part 1 or the MTD/RP2D minus one dose level depending on which arm (see Table 10). The dose of T(kK183C+K290C)-vc0101 ADC can be escalated or de-escalated based on the mTPI design and the DLT criteria and emerging data if indicated.

The Recommended Phase 2 Dose (RP2D) is the dose chosen for further investigation based on Phase 1 study results. If the MTh proves to be clinically feasible for long-term administration in a reasonable number of patients, then this dose usually becomes the RP2D. Further experience with the MTD may result in a RP2D dose lower than the MTD.

B. Patient Population

Key inclusion criteria for Part 2 includes: adult patients (age ≥18 years) with:

Arm A: Breast Cancer: Histological or cytological diagnosis of advanced/unresectable or metastatic HER2 positive (+) BC. Patients categorized as HER2 positive must be refractory to or have progressed on or are intolerant of established therapies known to provide clinical benefit in HER2+ breast cancer including herceptin, pertuzumab and ado-trastuzumab emtansine (T-DM1), either in combination or as a single agent, unless not indicated per local standard of care practice. Prior treatment on other monoclonal HER2 targeted therapies including margetuximab or trastuzumab deruxtecan (DS-8201) is allowed.

Arm B: Breast Cancer: Histological or cytological diagnosis of advanced/unresectable or metastatic hormone receptor positive (HR+), HER2 IHC2+/ISH negative (−) or equivocal. Patients categorized as HR+ (including documentation of estrogen receptor (ER) positive and/or progesterone receptor positive tumor (≥1% positive stained cells) based on most recent tumor biopsy utilizing an assay consistent with local standards) and HER2 IHC2+/ISH negative (−) or equivocal and must be refractory to or have progressed on or are intolerant of established therapies known to provide clinical benefit in HR+ breast cancer including anti hormone therapies and CDK (cyclin-dependent kinase) 4/6 inhibitors unless not indicated or allowed per local standard of care practice.

Arm C: Gastric Cancer: Histological or cytological diagnosis of advanced/unresectable or metastatic HER2+ and HER2 IHC2+/ISH negative (−) or equivocal adenocarcinoma of the stomach or esophagogastric junction. Patients must be refractory to or have progressed on or are intolerant of treatment with trastuzumab plus cisplatin/5-FU (fluorouracil) based regimen or standard therapy for primary (1st line) treatment of adenocarcinoma of the stomach or esophagogastric junction (gastric or gastroesophageal cancer).

Previous HER2 positive test results (Arm A and HER2+ patients in Arm C), using a Food and Drug Administration (FDA) approved or locally validated test will be accepted.

Arm D: NSCLC: Histological or cytological documented diagnosis of advanced NSCLC. Patients must be refractory to or have progressed on or are intolerant to treatment with an anti-PD-1 (programmed cell death protein 1)/programmed death ligand 1 (PD-L1) checkpoint inhibitor per standard therapy: Unless not indicated, patients must have been treated with anti-PD-1/L1 in combination with chemotherapy or as a monotherapy when PD-L1 expression ≥1% [Tumor Proportion Score ≥1%]. Patients with EGFR mutations and ALK rearrangements must have received a prior EGFR and ALK targeted therapy, respectively. If the tumor is T790M mutation positive NSCLC, the patient must have received osimertinib. Patients with ROS1 mutation-positive tumors must have received prior crizotinib.

Patients were excluded from this study if they met the following key exclusion criteria: Patients with known symptomatic brain metastases requiring steroids, and major surgery or systemic anticancer therapy within 4 weeks of starting treatment.

C. Treatment Schedule

Part 2B: T(kK183C+K290C)-vc0101 ADC Single Agent Dose Expansion

Part 2 dose expansion will evaluate T(kK183C+K290C)-vc0101 ADC administered at the MTD/RP2D in 21 days cycles as a single agent in four separate dose expansion arms as described herein (Arm A, B, C and D).

Part 2A: T(kK183C+K290C)-vc0101 ADC Combination Dose Finding

After the single-agent T(kK183C+K290C)-vc0101 ADC MTD/RP2D has been determined in Part 1, enrollment will be initiated into Part 2A in parallel with the Part 2 single agent dose expansion.

Part 2A will evaluate the T(kK183C+K290C)-vc0101 ADC MTD/RP2D dose in combination with pertuzumab±docetaxel (Arm 1 and Arm 2) and T(kK183C+K290C)-vc0101 ADC plus palbociclib and letrozole (Arm 3) in independent arms in women with HER2+ BC and HR+ HER2− mBC, respectively. It is anticipated that 3-6 patients will be enrolled in each arm of Part 2A and each Arm will have at least 3 DLT evaluable participants. The purpose of this portion of the study is to evaluate the safety and preliminary anti-tumor activity of T(kK183C+K290C)-vc0101 ADC in the patient populations described below:

Arm 1 and Arm 2: “First Line (1L) MBC”: HER2+; and

Arm 3: “First Line (1L) MBC”: HR+ HER2− mBC, with either failure of adjuvant treatment, or de novo MBC; with no prior exposure to CDK4/6 inhibitors.

Dose and Schedule:

T(kK183C+K290C)-vc0101 ADC will be administered as an IV infusion every 21 days (Q3W) and the combination drugs per Arm will be administered based on Table 10. Proposed Dose Levels for Part 2A Dose Finding Combination Arms.

TABLE 10
Proposed Dose Levels for Part 2A Dose Finding Combination Arms

| | Components | | | | |
|---|---|---|---|---|---|
| | T(kK183C+K290C)- | | | | |
| | vc0101ADC | | | | |
| | Starting Dose from Part 1 | Pertuzumab | Docetaxel | Palbociclib | Letrozole |
| Arm | (IV mg/kg Q3W) | (IV mg Q3W) | (IV mg/m2 Q3W) | (mg/day) ^{§} | (mg/day) |
| 1 | MTD/RP2D | C1D1: 840 mg | | | |
| | | D1 subsequent | | | |
| | | cycles: 420 mg | | | |
| 2 | MTD/RP2D-1 | C1D1: 840 mg | 75-100 | | |
| | | D1 subsequent | mg/m² | | |
| | | cycles: 420 mg | | | |
| 3 | MTD/RP2D-1 | | | 125 | 125 |

^{§} 3 weeks on followed by 1 week off

Part 2B: T(kK183C+K290C)-vc0101 ADC Combination Dose Expansion
Part 2B/Arm 1 and Arm 2—T(kK183C+K290C)-vc0101 ADC in Combination with Pertuzumab±Docetaxel in HER2+Locally Advanced or mBC (first line setting)

T(kK183C+K290C)-vc0101 ADC will be evaluated in combination with pertuzumab plus or minus docetaxel at the dose determined in Part 2A in Arm 1 and Arm 2 respectively in patients with HER2+advanced or mBC. Patients who have not previously received systemic anti-cancer therapy in the advanced or metastatic setting will be enrolled. Each arm will enroll up to 30 patients.

Dosing Regimen: Pertuzumab+T(kK183C+K290C)-vc0101 ADC+/−Docetaxel (Table 11):
Pertuzumab 840 mg IV day 1 followed by 420 mg IV;
T(kK183C+K290C)-vc0101 ADC RP2D IV day 1
   Cycled every 21 days
Pertuzumab will be give first followed by T(kK183C+K290C)-vc0101 ADC Docetaxel 75 mg/m2 Q3W
   Part 2B/Arm 3—T(kK183C+K290C)-vc0101 ADC in Combination with Palbociclib plus Letrozole in HR+ HER2− or HER2lo Locally Advanced or mBC (first line setting)

T(kK183C+K290C)-vc0101 ADC will be evaluated in combination with palbociclib plus letrozole at the dose determined in Part 2A in patients with HR+HER2− advanced or mBC in patients. Patients who have not previously received systemic anti-cancer therapy in the advanced or metastatic setting will be enrolled. This arm will enroll up to 30 patients.

Dosing Regimen: Letrozole+palbociclib+NG HER2 ADC (Table 11):
Letrozole 2.5 mg PO QD on days 1-28;
Palbociclib 125 mg/kg PO QD for 3 weeks
   Letrozole and palbociclib repeat every 28 days;
NG HER2 ADC RP2D IV day 1
   Cycled every 14 days.

The dose of palbociclib and letrozole should occur at approximately the same time as start of infusion.

See Table 11 below for information on dose and schedule.

TABLE 11
Proposed Dose Levels for Part 2B Dose Expansion Combination Arms

| | Components | | | | |
|---|---|---|---|---|---|
| | T(kK183C+K290C)- | | | | |
| | vc0101 ADC | | | | |
| | RP2D Dose from | Pertuzumab | Docetaxel | Palbociclib | Letrozole |
| Arm | Part 2A (IV mg/kg Q3W) | (IV mg Q3W) | (IV mg/m2 Q3W) | (mg/day) ^{§} | (mg/day) |
| 1 | TBD | C1D1: 840 mg | | | |
| | | D l subsequent | | | |
| | | cycles: 420 mg | | | |
| 2 | TBD | C1D1: 840 mg | 75-100 | | |
| | | D l subsequent | mg/m² | | |
| | | cycles: 420 mg | | | |
| 3 | TBD | | | 125 | 125 |

^{§} 3 weeks on followed by 1 week off

### Example 3

Dosage Forms, Packaging and Administration of Investigational Product Supplies

T(kK183C+K290C)-vc0101 ADC

T(kK183C+K290C)-vc0101 ADC is presented as a powder for reconstitution and IV administration. Each vial contains 40 mg of T(kK183C+K290C)-vc0101 ADC , is sealed with a coated stopper and an overseal, and is labeled according to local regulatory requirements.

T(kK183C+K290C)-vc0101 ADC will be administered on Day 1 of each 21 day cycle. A cycle is defined as the time from Day 1 dose to the next Day 1 dose. If there are no treatment delays, a cycle will be 21 days. In addition, alternative dosing schedules may be evaluated.

T(kK183 C+K290C)-vc0101 ADC will be administered intravenously over approximately 60 minutes (±15 minutes) on an outpatient basis.

The decision to incorporate pre-medication in all patients will be made following discussions between the sponsor and the investigators. Patients should be pre-treated with acetaminophen and diphenhydramine (or other antihistamine) approximately 0.5 to 2 hours before each PF-06804103 administration.

Suggested starting doses are 650 mg to 1000 mg acetaminophen and 50 mg diphenhydramine (or equivalent of other antihistamine) IV or oral. Two additional doses of acetaminophen may be administered approximately every 4-6 hours after the initial pre-treatment or as needed

When combining with palbociclib and letrozole, the treatment schedule (cycle and day for treatment) for PF-06804103 should follow that of palbociclib.

Pertuzumab

Pertuzumab 420 mg concentrate for solution for infusion and is a clear to slightly opalescent, colourless to pale yellow, liquid. One 14 ml vial of concentrate contains 420 mg of pertuzumab at a concentration of 30 mg/ml.

Pertuzumab initial dose is 840 mg administered as a 60-minute intravenous infusion, followed every 3 weeks thereafter by 420 mg administered as a 30 to 60 minute intravenous infusion.

Docetaxel

Docetaxel is sterile, non-pyrogenic, and is available in single-dose vials containing 20 mg (0.5 mL) or 80 mg (2 mL) docetaxel (anhydrous) and requires dilution prior to use. A sterile, non-pyrogenic, single-dose diluent is supplied for that purpose. The diluent contains 13% ethanol in water for injection, and is supplied in vials.

Docetaxel will be administered intravenously at the starting dose of 75 mg/m2 every 3 weeks. The total docetaxel dose will be administered as a 1-hour IV infusion. The dose of docetaxel will be calculated using body surface area (mg/m2).

Docetaxel must be used in compliance with its local prescribing information which should be reviewed to ensure that appropriate patients are enrolled in the study.

All patients must receive prophylactic pre-medication in order to reduce the incidence and severity of fluid retention and hypersensitivity reactions as per Institution's practices. Suggested pre-medication regimen before each chemotherapy administration consists of oral dexamethasone 8 mg bid or equipotent doses of oral prednisone or prednisolone or methylprednisolone given for 3 days starting 1 day prior to docetaxel administration.

The total docetaxel dose will be administered on Day 1 of each cycle as a 1-hour infusion.

Palbociclib

Palbociclib will be supplied as 125 mg capsules in High Density Polyethylene (HDPE) bottles, labeled according to local regulatory requirements. The 100 mg, and 75 mg capsules will be available for dose reduction.

Patients should be instructed to swallow palbociclib capsules whole and not to manipulate or chew them prior to swallowing. No capsule should be ingested if it is broken, cracked, or otherwise not intact. Patients should be encouraged to take their dose at approximately the same time each day. Patients should be instructed to record daily administration in the patient diary.

Patients should take palbociclib with food. Palbociclib will be administered orally once a day for 21 days followed by 7 days off treatment for each 28-day cycle.

Letrozole

The recommended dose is one 2.5 mg tablet administered once a day, with or without meals, on a continuous basis on days 1 through 28.

1-25. (canceled)

26. A method for the treatment of a patient having cancer, comprising administering to the patient an effective amount of an anti-HER2 ADC at least twice every week, at least weekly (QW), at least every 2 weeks (Q2W), at least every 3 weeks (Q3W) or at least every 4 weeks (Q4W), wherein the anti-HER2 ADC comprises an anti-HER2 antibody conjugated to an anti-cancer drug.

27. The method of claim 26, wherein the anti-HER2 ADC is administered every 3 weeks (Q3W).

28. The method of claim 26 or 27, wherein the anti-HER2 ADC is administered at a dose of about 0.010 mg/kg to about 10 mg/kg, about 0.010 mg/kg to about 5 mg/kg, about 0.10 mg/kg to about 1 mg/kg or about 0.10 mg/kg to about 0.50 mg/kg.

29. The method of any one of claims 26 to 28, wherein the anti-HER2 ADC is administered at a dose of at least 0.10, 0.15, 0.20, 0.25, 0.30, 0.35, 0.40, 0.45, 0.50, 0.55, 0.60, 0.65, 0.70, 0.75, 0.80, 0.95, 1.00, 1.10, 1.20, 1.30, 1.40, 1.50, 2.00, 2.50, 2.70, 3.00, 3.50, 4.00, 4.50, 5.00, 5.50, 6.00 mg/kg.

30. The method of any one of claims 26 to 29, wherein the anti-HER2 ADC is administered at a dose of about 0.15 mg/kg, 0.50 mg/kg, 1.20 mg/kg, 2.00 mg/kg, 2.70 mg/kg, 3.00 mg/kg, 4.00 mg/kg, 5.00 mg/kg, or 6.00 mg/kg.

31. The method of any one of claims 26 to 30, wherein the anti-HER2 ADC is administered every 3 weeks (Q3W) at a dose of about 0.15 mg/kg, 0.50 mg/kg, 1.20 mg/kg, 2.00 mg/kg, 2.70 mg/kg, 3.00 mg/kg, 4.00 mg/kg, 5.00 mg/kg, or 6.00 mg/kg.

32. The method of claim 31, wherein the anti-HER2 ADC is administered every 3 weeks (Q3W) at a dose of about 3.00 mg/kg, 4.00 mg/kg, 5.00 mg/kg, or 6.00 mg/kg.

33. The method of claim 32, wherein the anti-HER2 ADC is administered every 3 weeks (Q3W) at a dose of about 4.00 mg/kg mg/kg.

34. The method of any one of claims 26-33, wherein the anti-HER2 ADC is administered intravenously, subcutaneously, intramuscularly, by bolus injection, intracerebrally or by sustained release.

35. (canceled)

36. The method of any one of claims 26 to 35, wherein the antibody comprises a VH CDR1 having the amino acid sequence shown in SEQ ID NO: 2, VH CDR2 having the amino acid sequence shown in SEQ ID NO: 3, and VH CDR3 having the amino acid sequence shown in SEQ ID NO: 4, and/or VL CDR1 having the amino acid sequence shown in SEQ ID NO: 8, VL CDR2 having the amino acid sequence shown in SEQ ID NO: 9, and VL CDR3 having the amino acid sequence shown in SEQ ID NO: 10.

37. The method of any one of claims 26 to 36, wherein the antibody comprises a heavy chain protein having the amino acid sequence shown in SEQ ID NO: 14 and a light chain protein having the amino acid sequence shown in SEQ ID NO: 16.

38. The method any one of claims 26 to 37, wherein the antibody is T(kK183C+K290C).

39. The method of any one of claims 26 to 39, wherein the anti-cancer drug is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3 -thiazol-2-yl)ethyl]amino}propyl]pyrrolidin-1-yl-}-5-methyl1-oxoheptan-4-yl]-N-methyl-L-valinamide (0101).

40. The method of any one of claims 26 to 39, wherein the anti-HER2 ADC further comprises a linker.

41. The method of claim 40, wherein the linker is maleimidocaproyl-valine-citrulline-p-aminobenzyloxycarbonyl (vc).

42. The method any one of claims 26 to 41, wherein the anti-HER2 ADC is T(cK183C+K290C)-vc0101.

43. The method of any one of claims 26 to 42, wherein the cancer is characterized by overexpression of HER2.

44. The method of any one of claims 26 to 42, wherein the cancer is hormone receptor positive.

45. The method of any one of claims 26 to 44, wherein the cancer is breast cancer, hormone receptor positive breast cancer, estrogen receptor and progesterone receptor negative breast cancer, triple negative breast cancer (TNBC), ovarian cancer, lung cancer, non-small cell lung cancer (NSCLC), gastric cancer, esophageal cancer, colorectal cancer, urothelial cancer, pancreatic cancer, salivary gland cancer and brain cancer or metastases thereof.

46. The method of claim 44, wherein the cancer is breast cancer, gastric cancer, or NSCLC.

47-50. (cancelled)
